(19) United States
(12) Patent Application Publication
(10) Pub. No.: US 2023/0193356 A1
(43) Pub. Date: Jun. 22, 2023

(54) SINGLE CELL COMBINATORIAL INDEXING FROM AMPLIFIED NUCLEIC ACIDS

(71) Applicants: THE BROAD INSTITUTE, INC., Cambridge, MA (US); MASSACHUSETTS INSTITUTE OF TECHNOLOGY, Cambridge, MA (US); THE GENERAL HOSPITAL CORPORATION, Boston, MA (US)

(72) Inventors: Aziz Al'Khafaji, Cambridge, MA (US); Paul Blainey, Cambridge, MA (US); Nir Hacohen, Boston, MA (US)

(73) Assignees: THE BROAD INSTITUTE, INC., Cambridge, MA (US); MASSACHUSETTS INSTITUTE OF TECHNOLOGY, Cambridge, MA (US); THE GENERAL HOSPITAL CORPORATION, Boston, MA (US)

(21) Appl. No.: 18/000,222

(22) PCT Filed: Jun. 7, 2021
(86) PCT No.: PCT/US21/36214

§ 371 Date: Nov. 29, 2022

Related U.S. Application Data
(60) Provisional application No. 63/036,138, filed on Jun. 8, 2020.

(51) Int. Cl. C12Q 1/6806

(52) U.S. Cl. CPC C12Q 1/6806

(57) ABSTRACT

The present disclosure relates to compositions and methods for single-cell nucleic acid sequencing, and specifically provides for pre-amplifying target nucleic acids in a manner that allows for more proportionate detection of all target nucleic acids, including low prevalence/abundance RNAs, from individual cells. The disclosure also provides for application of a series of barcoding steps to associate cell-specific identifiers (IDs) to the targeted nucleotide sequences, and ultimately provides for increased throughput capacity and greater accuracy of single-cell nucleic acid sequencing. Certain aspects of the present disclosure also provide for improved quantitative detection of nucleic acid sequence barcodes, which in embodiments allows for highly sensitive quantitative detection of barcoded antibody levels and/or highly sensitive quantitative detection of barcoded antibody-bound protein levels (e.g., where specific antibodies are labeled with a barcoded oligonucleotide that is specific to each barcoded antibody's target. In such approaches, the oligonucleotide barcode can serve as a target nucleic acid sequence for the capture probes of the instant disclosure. Compositions, methods and kits related to specific combinations of capture probes are also provided.

### CROSS-REFERENCE TO RELATED APPLICATION

This application claims the benefit of U.S. Provisional Application No. 63/036,138, filed Jun. 8, 2020, entitled “Single Cell Combinatorial Indexing from Amplified Nucleic Acids.” The entire contents of the aforementioned application are incorporated herein by reference.

### STATEMENT REGARDING FEDERALLY SPONSORED RESEARCH

This invention was made with government support under Grant No. P50 HG006193 awarded by the National Institutes of Health. The government has certain rights in the invention.

### FIELD OF THE INVENTION

The invention relates generally to methods and compositions for single-cell sequencing.

### BACKGROUND OF THE INVENTION

Previously described single-cell sequencing approaches have lacked the capacity to profile greater than a million (1E6) cells with high RNA capture efficiency. In particular, droplet-based approaches suffer scaling limitations due to the inefficiencies of single-cell/bead barcode encapsulation, while combinatorial indexing approaches exhibit significant data quality drop-off during higher rounds of indexing. Known droplet based single-cell approaches therefore offer a modest throughput with good target capture, while combinatorial indexing approaches allow for higher throughput at the cost of sparse target capture. A need therefore exists for single-cell sequencing methods capable of directly targeting and amplifying RNA sequences of interest at high throughput (e.g., profiling greater than 1E6 cells) with improved accuracy, efficiency and specificity.

### BRIEF SUMMARY OF THE INVENTION

The current disclosure relates, at least in part, to compositions and methods for performing single-cell sequencing upon nucleic acids, particularly upon mRNAs, snRNAs, lncRNAs, siRNAs, and gRNAs—and in certain embodiments upon DNA (e.g., in applications such as CITE-Seq (Cellular Indexing of Transcriptomes and Epitopes by Sequencing), where sequence-specific measurement of DNA abundance (i.e. DNA-indexed antibodies) can serve as a proxy for corresponding protein abundance)—in a tissue or cell sample (including, e.g., in nuclei), with high accuracy, efficiency, specificity, and cell throughput. Here, it has been discovered that previously described single cell sequencing methods' inefficiencies in target capture can now be mitigated as disclosed herein by amplification of the target prior to combinatorial indexing. The currently disclosed enhanced workflow represents a robust ultra-high throughput single-cell sequencing method that allows for efficient target capture. Specifically contemplated applications for such improved high accuracy, efficiency, specificity, and cell throughput of single-cell sequencing—e.g., single-cell RNA sequencing—include, but are not limited to, single or combinatorial gene expression profiles associated with disease phenotypes, evaluation of nucleotide therapy delivery and integration into tissue or cells, including, e.g., delivery of siRNAs, cellular CRISPR/Cas9 gRNAs, expression of CRISPR/Cas9 or TALEN plasmid(s), viral vectors (e.g., AAV), and expression of vectors/plasmids in general, as well as applications in which nucleic acid abundance is measured as a proxy for some other measurement, such as in CITE-Seq, where nucleic acid-barcoded antibodies can be more precisely quantitated using the approach(es) described herein, as a proxy for measurement of bound corresponding cellular protein levels. The instant disclosure also provides sufficient sensitivity to detect snRNAs and lncRNAs, thereby facilitating the elucidation of biological pathways heretofore inaccessible. Precise evaluation of low frequency RNA species—including low abundance mRNAs, snRNAs, lncRNAs, siRNAs, and gRNAs—is also provided. Similarly, precise evaluation of low frequency DNA sequences—as occur, e.g., in CITE-Seq and other approaches reliant upon quantitation of DNA abundance—is provided. A wide range of diagnostic, therapeutic and research applications are therefore contemplated.

In one aspect, the instant disclosure provides a method for performing single-cell nucleic acid sequencing upon cells of a tissue sample, the method involving: (i) obtaining a tissue sample from a subject; (ii) permeabilizing cells of the tissue sample; (iii) contacting the permeabilized cells of the tissue sample with a padlock probe having a sequence complementary to a target nucleic acid sequence, thereby producing a padlock probe bound to the target nucleic acid sequence; (iv) contacting the treated cells with a reverse transcriptase (for RNA target nucleic acid sequences, as well as many DNA target nucleic acid sequences) or a polymerase (for certain DNA target nucleic acid sequences), thereby capturing the target nucleic acid sequence (i.e., the complement of the target nucleic acid sequence bound by the padlock probe) on the padlock probe; (v) performing ligation (contacting the target nucleic acid sequence on the padlock probe with ligase), thereby circularizing the padlock probe bound to the target nucleic acid sequence; (vi) performing rolling circle amplification (RCA) upon the circularized padlock probe, thereby creating a linear repeating sequence (LRS) that includes the target nucleic acid sequence; (vii) contacting said LRS with a primer having an LRS complement sequence and an index adaptor sequence; (viii) subjecting the treated cells of the tissue sample to combinatorial indexing, thereby generating an extended primer that includes the LRS complement sequence, the adaptor sequence, and a barcode sequence capable of identifying the cell of origin; and (ix) identifying a polynucleotide sequence of the extended primer, thereby obtaining single cell nucleic acid sequencing data from the tissue sample.

In one embodiment, the target nucleic acid sequence includes a target RNA sequence or complement thereof.

In certain embodiments, the padlock probe includes a unique molecular identifier (UMI). Optionally, the UMI is between 8 and 20 nucleotides in length.

In some embodiments, the barcode sequence is between 6 and 20 nucleotides in length.

In embodiments, the target nucleic acid sequence includes or is a RNA sequence. Optionally, the RNA sequence includes or is a mRNA, a snRNA, a lcRNA, a siRNA and/or a gRNA.

In one embodiment, the target nucleic acid sequence includes a mRNA or other nucleic acid sequence. Optionally one or more such a sequence is that of a pathway and/or gene of FIG. 6.

In certain embodiments, the target nucleic acid sequence includes a DNA barcode sequence. Optionally, the DNA barcode sequence identifies and/or is attached to an antibody. In a related embodiment, detection of the DNA barcode sequence identifies antibody abundance and/or levels of a protein bound by the barcode-associated antibody. Optionally, the method is performed to quantify target protein levels in a CITE-Seq and/or REAP-Seq process.

In some embodiments, combinatorial indexing is applied in combination with cell splitting. Optionally, combinatorial indexing is applied in combination with between 1 and 10 iterations of cell splitting.

In certain embodiments, combinatorial indexing involves use of a microfluidic chamber.

In some embodiments, RCA employs a DNA polymerase.

In certain embodiments, single cell nucleic acid sequencing data is obtained from between about 1,000,000 and about cells 1×10¹² in a single run.

In some embodiments, the target RNA is a low abundance RNA, optionally a RNA that is present at less than ten copies in a single cell, optionally less than nine copies in a single cell, optionally less than eight copies in a single cell, optionally less than seven copies in a single cell, optionally less than six copies in a single cell, optionally less than five copies in a single cell, optionally less than four copies in a single cell, optionally less than three copies in a single cell, optionally less than two copies in a single cell, optionally at one copy in a single cell.

In certain embodiments, the polymerase is a non-strand displacing DNA polymerase. Optionally, the non-strand displacing DNA polymerase is Q5® High-Fidelity DNA Polymerase, Phusion® High-Fidelity DNA Polymerase, KAPA HiFi DNA Polymerase, Pfu DNA polymerase, KOD DNA polymerase, T4 DNA polymerase, T7 DNA polymerase, and/or an exonuclease deficient variant of Taq (TaqIT).

Another aspect of the instant disclosure provides an improved method for obtaining quantitative nucleic acid sequence data, the method involving: (i) contacting a sample that includes a target nucleic acid sequence with a padlock probe having a sequence complementary to the target nucleic acid sequence, thereby generating a padlock probe bound to the target nucleic acid sequence; (ii) contacting the sample with a reverse transcriptase (for RNA target nucleic acid sequences, as well as many DNA target nucleic acid sequences) or a polymerase (for certain DNA target nucleic acid sequences), thereby capturing the target nucleic acid sequence (i.e., the complement of the target nucleic acid sequence bound by the padlock probe) on the padlock probe; (iii) contacting the target nucleic acid sequence on the padlock probe with a ligase, thereby circularizing the padlock probe bound to the target nucleic acid sequence; (iv) performing rolling circle amplification (RCA) upon the circularized padlock probe, thereby generating a linear repeating sequence (LRS) that includes the target nucleic acid sequence; and (v) obtaining a sequence of the LRS and optionally correlating the sequence of the LRS with a single target nucleic acid and/or single cell of origin, thereby obtaining quantitative nucleic acid sequence data.

An additional aspect of the instant disclosure provides a composition that includes a plurality of padlock probes targeting two or more genes and/or RNAs of a pathway and/or gene of FIG. 6.

A further aspect of the instant disclosure provides a kit that includes a plurality of padlock probes targeting two or more genes and/or RNAs of a pathway and/or gene of FIG. 6 and instructions for its use.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

By “control” or “reference” is meant a standard of comparison. Methods to select and test control samples are within the ability of those in the art. Determination of statistical significance is within the ability of those skilled in the art, e.g., the number of standard deviations from the mean that constitute a positive result.

As used herein, the term “different”, when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different for the two or more molecules while also having a universal sequence portion that is the same on the two or more molecules.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, single cell and/or enhanced sensitivity nucleic acid sequencing refers to methods for measuring the sequence of cellular or other types of nucleic acids in a sample (optionally, barcodes, e.g., barcoded proteins, e.g., barcoded antibodies) and identifying the individual cell(s) and/or barcode-associated moiety (e.g., protein(s)) from which the cellular and/or sample nucleic acid(s) were obtained. Similarly, single cell RNA sequencing refers to methods for measuring the sequence of cellular RNA(s) (optionally, transcripts) and identifying the individual cell(s) from which the cellular RNA(s) were obtained.

As used herein, a “padlock probe” refers to a nucleic acid probe that on its 5′ and 3′ ends hybridizes to the 5′ and 3′ ends of a target sequence, wherein the probe circularizes upon sequence extension and ligation. Padlock probes are described in U.S. Pat. No. 5,854,033 (Lizardi), WO99/49079 (Landegren) and U.S. Pat. No. 5,871,921 (Landegren & Kwiatkowski). A version of the padlock probe known as the inversion probe is described in U.S. Pat. No. 6,858,412 (Willis et al.). Inversion probes are padlock probes containing a cleavage site in the probe backbone, allowing the circularized probe to be cleaved to form a linear product, which may then be amplified and detected. In some embodiments the padlock probe can capture genetic information directly from a target RNA sequence by using a non-strand displacing reverse transcriptase such as RTX or using reverse transcriptase reactions that prevent or minimize strand displacement with widely used viral reverse transcriptases such as M-HLV. For certain DNA target nucleic acid sequences, a DNA polymerase (e.g., Q5® High-Fidelity DNA Polymerase, Phusion® High-Fidelity DNA Polymerase, KAPA HiFi DNA Polymerase, Pfu DNA polymerase, KOD DNA polymerase, T4 DNA polymerase, T7 DNA polymerase, and/or an exonuclease deficient variant of Taq (TaqIT)) can be used for similar purpose. Once gap filled and ligated with the appropriate ligase, the circularized padlock can then undergo RCA. In certain embodiments, a padlock probe harbors an identifying sequence, such as a unique molecular identifier (UMI) sequence.

As used herein, “rolling circle amplification” (RCA) refers to a nucleic acid amplifying process that produces tens to hundreds or thousands of copies of a circularized product in a single molecule, thereby effectively amplifying the circularized product and making it relatively easy to detect individually, e.g., even where probe hybridization efficiencies to any individual target sequence might be low. A suitable nucleic acid template that includes a target sequence can be produced using techniques known in the art and as specifically described herein. Exemplified nucleic acid templates include circular nucleic acids, particularly a circular nucleic acid having a double-stranded central region and two single-stranded hairpin end regions (that is, loops connecting the two complementary strands of the double-stranded region). The double-stranded central region typically comprises the target region. As will be appreciated, the term circular, when referring to the strand configuration, merely denotes a strand of a nucleic acid that includes no terminal nucleotides, and does not necessarily denote any geometric configuration. In some embodiments, the RCA product contains UMIs and the captured RNA information is now an improved substrate for combinatorial indexing as multiple copies of the RNA molecule are made.

As used herein “combinatorial indexing” refers to the process of identifying cells by applying a series of barcode sequences that in combination form a cell identifying sequence. Specifically, “split-pool barcoding” refers to a process of combinatorial indexing by which cells are split into microwells and exposed to well-specific barcode sequences, that in combination, form a cell identifying sequence. In certain embodiments of the instant disclosure, split-pool barcoding can performed between one and eight times (i.e., 1, 2, 3, 4, 5, 6, 7 and/or 8 times).

As used herein, SPLiT-seq refers to the single-cell sequencing process by which individual transcriptomes are uniquely labeled by passing a suspension of formaldehyde-fixed cells or nuclei through four rounds of combinatorial barcoding. In the first round of barcoding, cells are split and distributed into a microwell plate, and cDNA is generated with an in-cell reverse transcription (RT) reaction using well-specific barcoded primers. Each well can contain a different biological sample, thereby enabling multiplexing of up to 96 samples in a single experiment. After this step, cells from all wells are pooled and redistributed into a new 96-well plate, where an in-cell ligation reaction appends a second well-specific barcode to the cDNA. The third-round barcode, which also contains a unique molecular identifier (UMI), is then appended with another round of pooling, splitting, and ligation. After three rounds of barcoding, the cells are pooled and split into sublibraries, and sequencing barcodes are introduced by polymerase chain reaction (PCR). This final step provides a fourth barcode, while also making it possible to sequence different numbers of cells in each sublibrary. After sequencing, each transcriptome is assembled by combining reads containing the same four-barcode combination

As used herein, Sci-seq refers generally to single-cell combinatorial indexing and sequencing, including DNA, RNA, or protein sequencing.

As used herein, a unique molecular identifier (UMI) of e.g. 8-12 or more random nucleotides can be incorporated into the padlock probe, to allow an ex post facto identification of all PCR products with the same UMI to one probe. The chance of 2 different padlock probes having the same 12 nucleotide UMI is (1/4)⁽¹²⁾=1:16,777,216, which tends to make UMIs unique for each probe.

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), multiple displacement amplification (MDA), ligation extension, or ligation chain reaction. An amplicon can be a nucleic acid molecule having a single copy of a particular nucleotide sequence (e.g. a PCR product) or multiple copies of the nucleotide sequence (e.g. a concatameric product of RCA). A first amplicon of a target nucleic acid is typically a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

As used herein, the term “array” refers to a population of features or sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate.

As used herein, the term “barcode sequence” is intended to mean a series of nucleotides in a nucleic acid that can be used to identify the nucleic acid, a characteristic of the nucleic acid (e.g., the identity), or a manipulation that has been carried out on the nucleic acid. The barcode sequence can be a naturally occurring sequence or a sequence that does not occur naturally in the organism from which the barcoded nucleic acid was obtained. A barcode sequence can be unique to a single nucleic acid species in a population or a barcode sequence can be shared by several different nucleic acid species in a population. By way of further example, each nucleic acid probe in a population can include different barcode sequences from all other nucleic acid probes in the population. Alternatively, each nucleic acid probe in a population can include different barcode sequences from some or most other nucleic acid probes in a population. For example, each probe in a population can have a barcode that is present for several different probes in the population even though the probes with the common barcode differ from each other at other sequence regions along their length. In particular embodiments, one or more barcode sequences that are used with a biological specimen (e.g., a tissue sample) are not present in the genome, transcriptome or other nucleic acids of the biological specimen. For example, barcode sequences can have less than 80%, 70%, 60%, 50% or 40% sequence identity to the nucleic acid sequences in a particular biological specimen.

As used herein, the term “extend,” when used in reference to a nucleic acid, is intended to mean addition of at least one nucleotide or oligonucleotide to the nucleic acid. In particular embodiments one or more nucleotides can be added to the 3′ end of a nucleic acid, for example, via polymerase catalysis (e.g. DNA polymerase, RNA polymerase or reverse transcriptase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g. ligase catalysis) methods. A nucleic acid can be extended in a template directed manner, whereby the product of extension is complementary to a template nucleic acid that is hybridized to the nucleic acid that is extended.

As used herein, the term “reverse transcriptase” refers to an enzyme used to generate complementary DNA (cDNA) from an RNA template. Reverse transcriptases (RTs) commonly used in the art include the non-strand displacing transcriptase RTX, and the viral reverse transcriptase M-MLV.

As used herein, “amplify”, “amplifying” or “amplification reaction” and their derivatives, refer generally to any action or process whereby at least a portion of a nucleic acid molecule is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. Amplification optionally includes linear or exponential replication of a nucleic acid molecule. In some embodiments, such amplification can be performed using isothermal conditions; in other embodiments, such amplification can include thermocycling. In some embodiments, the amplification is a multiplex amplification that includes the simultaneous amplification of a plurality of target sequences in a single amplification reaction. In some embodiments, “amplification” includes amplification of at least some portion of DNA and RNA based nucleic acids alone, or in combination. The amplification reaction can include any of the amplification processes known to one of ordinary skill in the art. In some embodiments, the amplification reaction includes polymerase chain reaction (PCR) amplifying one or more nucleic acid sequences. Such amplification can be linear or exponential. In some embodiments, the amplification conditions can include isothermal conditions or alternatively can include thermocycling conditions, or a combination of isothermal and thermocycling conditions. In some embodiments, the conditions suitable for amplifying one or more nucleic acid sequences include polymerase chain reaction (PCR) conditions. Typically, the amplification conditions refer to a reaction mixture that is sufficient to amplify nucleic acids such as one or more target sequences flanked by a universal sequence, or to amplify an amplified target sequence ligated to one or more adaptors. Generally, the amplification conditions include a catalyst for amplification or for nucleic acid synthesis, for example a polymerase; a primer that possesses some degree of complementarity to the nucleic acid to be amplified; and nucleotides, such as deoxyribonucleotide triphosphates and ribononucleic triphosphates to promote extension of the primer once hybridized to the nucleic acid. The amplification conditions can require hybridization or annealing of a primer to a nucleic acid, extension of the primer and a denaturing step in which the extended primer is separated from the nucleic acid sequence undergoing amplification. As used herein, the term “polymerase chain reaction” (“PCR”) refers to the method of Mullis U.S. Pat. Nos. 4,683,195 and 4,683,202, which describe a method for increasing the concentration of a segment of a polynucleotide of interest. As used herein, “amplified target sequences” and its derivatives, refers generally to a nucleic acid sequence produced by the amplifying the target sequences using target-specific primers and the methods provided herein. The amplified target sequences may be either of the same sense (i.e. the positive strand) or antisense (i.e., the negative strand) with respect to the target sequences.

As used herein, the terms “ligating”, “ligation” and their derivatives refer generally to the process for covalently linking two or more molecules together, for example covalently linking two or more nucleic acid molecules to each other. In some embodiments, ligation includes joining nicks between adjacent nucleotides of nucleic acids. In some embodiments, ligation includes forming a covalent bond between an end of a first and an end of a second nucleic acid molecule. In some embodiments, the ligation can include forming a covalent bond between a 5′ phosphate group of one nucleic acid and a 3′ hydroxyl group of a second nucleic acid thereby forming a ligated nucleic acid molecule. Generally, for the purposes of this disclosure, an amplified target sequence can be ligated to an adaptor to generate an adaptor-ligated amplified target sequence.

As used herein, “ligase” and its derivatives, refers generally to any agent capable of catalyzing the ligation of two substrate molecules. In some embodiments, the ligase includes an enzyme capable of catalyzing the joining of nicks between adjacent nucleotides of a nucleic acid. In some embodiments, the ligase includes an enzyme capable of catalyzing the formation of a covalent bond between a 5′ phosphate of one nucleic acid molecule to a 3′ hydroxyl of another nucleic acid molecule thereby forming a ligated nucleic acid molecule. Suitable ligases may include, but are not limited to, T4 DNA ligase, T4 RNA ligase, and E. coli DNA ligase.

As used herein, “ligation conditions” and its derivatives, generally refers to conditions suitable for ligating two molecules to each other.

As used herein, the term “next-generation sequencing” or “NGS” can refer to sequencing technologies that have the capacity to sequence polynucleotides at speeds that were unprecedented using conventional sequencing methods (e.g., standard Sanger or Maxam-Gilbert sequencing methods). These unprecedented speeds are achieved by performing and reading out thousands to millions of sequencing reactions in parallel. NGS sequencing platforms include, but are not limited to, the following: Massively Parallel Signature Sequencing (Lynx Therapeutics); 454 pyrosequencing (454 Life Sciences/Roche Diagnostics); solid-phase, reversible dye-terminator sequencing (Solexa/Illumina™); SOLiD™ technology (Applied Biosystems); Ion semiconductor sequencing (Ion Torrent™); and DNA nanoball sequencing (Complete Genomics). Descriptions of certain NGS platforms can be found in the following: Shendure, er al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3):333-43; and Zhang et al., “The impact of next-generation sequencing on genomics”, J Genet Genomics, 201, 38(3): 95-109.

As used herein, the terms “nucleic acid” and “nucleotide” are intended to be consistent with their use in the art and to include naturally occurring species or functional analogs thereof. Particularly useful functional analogs of nucleic acids are capable of hybridizing to a nucleic acid in a sequence specific fashion or capable of being used as a template for replication of a particular nucleotide sequence.

Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g. found in deoxyribonucleic acid (DNA)) or a ribose sugar (e.g. found in ribonucleic acid (RNA)). A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine, thymine, cytosine or guanine and a ribonucleic acid can have one or more bases selected from the group consisting of uracil, adenine, cytosine or guanine. Useful non-native bases that can be included in a nucleic acid or nucleotide are known in the art. The terms “probe” or “target,” when used in reference to a nucleic acid or sequence of a nucleic acid, are intended as semantic identifiers for the nucleic acid or sequence in the context of a method or composition set forth herein and does not necessarily limit the structure or function of the nucleic acid or sequence beyond what is otherwise explicitly indicated.

A target nucleic acid may be essentially any nucleic acid of known or unknown sequence. It may be, for example, any RNA transcript coding or non-coding including but not limited to: mRNAs, snRNAs, lncRNAs, siRNAs, and gRNAs. A target nucleic acid may be a fragment of genomic DNA (e.g., chromosomal DNA), extra-chromosomal DNA such as a plasmid, cell-free DNA, or cDNA. Sequencing may result in determination of the sequence of the whole, or a part of the target molecule. The targets can be derived from a primary nucleic acid sample, such as a cytoplasm or nucleus. In one embodiment, the targets can be processed into templates suitable for amplification by the placement of universal sequences at one or both ends of each target fragment. The targets may be obtained from a primary RNA sample by reverse transcription into cDNA. The targets may be obtained by padlock probe hybridization followed by rolling circle amplification. Targeted sequencing uses selection and isolation of genes or regions or proteins of interest, typically by either PCR amplification (e.g., region-specific primers) or hybridization-based capture method (e.g., use of a capture probe) or antibodies. Targeted enrichment can occur at various stages of the method. For instance, a targeted RNA representation can be obtained using target specific primers in the reverse transcription step or hybridization based enrichment of a subset out of a more complex library. Targeted sequencing can include any of the enrichment processes known to one of ordinary skill in the art.

The terms “P5” and “P7” may be used when referring to a universal capture sequence or a capture oligonucleotide. The terms “P5′” (P5 prime) and “P7′” (P7 prime) refer to the complement of P5 and P7, respectively. It will be understood that any suitable universal capture sequence or a capture oligonucleotide can be used in the methods presented herein, and that the use of P5 and P7 are exemplary embodiments only. Uses of capture oligonucleotides such as P5 and P7 or their complements on flow cells are known in the art, as exemplified by the disclosures of WO 2007/010251, WO 2006/064199, WO 2005/065814, WO 2015/106941, WO 1998/044151, and WO 2000/018957. For example, any suitable forward amplification primer, whether immobilized or in solution, can be useful in the methods presented herein for hybridization to a complementary sequence and amplification of a sequence. Similarly, any suitable reverse amplification primer, whether immobilized or in solution, can be useful in the methods presented herein for hybridization to a complementary sequence and amplification of a sequence. One of skill in the art will understand how to design and use primer sequences that are suitable for capture and/or amplification of nucleic acids as presented herein.

As used herein, the term “primer” and its derivatives refer generally to any nucleic acid that can hybridize to a target sequence of interest. Typically, the primer functions as a substrate onto which nucleotides can be polymerized by a polymerase or to which a nucleotide sequence such as an index can be ligated; in some embodiments, however, the primer can become incorporated into the synthesized nucleic acid strand and provide a site to which another primer can hybridize to prime synthesis of a new strand that is complementary to the synthesized nucleic acid molecule. The primer can include any combination of nucleotides or analogs thereof. In some embodiments, the primer is a single-stranded oligonucleotide or polynucleotide. The terms “polynucleotide” and “oligonucleotide” are used interchangeably herein to refer to a polymeric form of nucleotides of any length, and may include ribonucleotides, deoxyribonucleotides, analogs thereof, or mixtures thereof. The terms should be understood to include, as equivalents, analogs of either DNA, RNA, or cDNA and double stranded polynucleotides. The term as used herein also encompasses cDNA, that is complementary or copy DNA produced from a RNA template, for example by the action of reverse transcriptase. This term refers only to the primary structure of the molecule.

As used herein, the term “random” can be used to refer to the spatial arrangement or composition of locations on a surface. For example, there are at least two types of order for an array described herein, the first relating to the spacing and relative location of features (also called “sites”) and the second relating to identity or predetermined knowledge of the particular species of molecule that is present at a particular feature. Accordingly, features of an array can be randomly spaced such that nearest neighbor features have variable spacing between each other. Alternatively, the spacing between features can be ordered, for example, forming a regular pattern such as a rectilinear grid or hexagonal grid. In another respect, features of an array can be random with respect to the identity or predetermined knowledge of the species of analyte (e.g., nucleic acid of a particular sequence) that occupies each feature independent of whether spacing produces a random pattern or ordered pattern. An array set forth herein can be ordered in one respect and random in another. For example, in some embodiments set forth herein a surface is contacted with a population of nucleic acids under conditions where the nucleic acids attach at sites that are ordered with respect to their relative locations but ‘randomly located’ with respect to knowledge of the sequence for the nucleic acid species present at any particular site. Reference to “randomly distributing” nucleic acids at locations on a surface is intended to refer to the absence of knowledge or absence of predetermination regarding which nucleic acid will be captured at which location (regardless of whether the locations are arranged in an ordered pattern or not).

As used herein, the term “biological specimen” is intended to mean one or more cell, tissue, organism or portion thereof. A biological specimen can be obtained from any of a variety of organisms. Exemplary organisms include, but are not limited to, a mammal such as a rodent, mouse, rat, rabbit, guinea pig, ungulate, horse, sheep, pig, goat, cow, cat, dog, primate (i.e. human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum. Target nucleic acids can also be derived from a prokaryote such as a bacterium, Escherichia coli, Staphylococci or Mycoplasma pneumoniae; an archae; a virus such as Hepatitis C virus or human immunodeficiency virus; or a viroid. Specimens can be derived from a homogeneous culture or population of the above organisms or alternatively from a collection of several different organisms, for example, in a community or ecosystem.

As used herein, the term “cell type” is intended to identify cells based on morphology, phenotype, developmental origin or other known or recognizable distinguishing cellular characteristic. A variety of different cell types can be obtained from a single organism (or from the same species of organism). Exemplary cell types include, but are not limited to, gametes (including female gametes, e.g., ova or egg cells, and male gametes, e.g. sperm), ovary epithelial, ovary fibroblast, testicular, urinary bladder, immune cells, B cells, T cells, natural killer cells, dendritic cells, cancer cells, eukaryotic cells, stem cells, blood cells, muscle cells, fat cells, skin cells, nerve cells, bone cells, pancreatic cells, endothelial cells, pancreatic epithelial, pancreatic alpha, pancreatic beta, pancreatic endothelial, bone marrow lymphoblast, bone marrow B lymphoblast, bone marrow macrophage, bone marrow erythroblast, bone marrow dendritic, bone marrow adipocyte, bone marrow osteocyte, bone marrow chondrocyte, promyeloblast, bone marrow megakaryoblast, bladder, brain B lymphocyte, brain glial, neuron, brain astrocyte, neuroectoderm, brain macrophage, brain microglia, brain epithelial, cortical neuron, brain fibroblast, breast epithelial, colon epithelial, colon B lymphocyte, mammary epithelial, mammary myoepithelial, mammary fibroblast, colon enterocyte, cervix epithelial, breast duct epithelial, tongue epithelial, tonsil dendritic, tonsil B lymphocyte, peripheral blood lymphoblast, peripheral blood T lymphoblast, peripheral blood cutaneous T lymphocyte, peripheral blood natural killer, peripheral blood B lymphoblast, peripheral blood monocyte, peripheral blood myeloblast, peripheral blood monoblast, peripheral blood promyeloblast, peripheral blood macrophage, peripheral blood basophil, liver endothelial, liver mast, liver epithelial, liver B lymphocyte, spleen endothelial, spleen epithelial, spleen B lymphocyte, liver hepatocyte, liver, fibroblast, lung epithelial, bronchus epithelial, lung fibroblast, lung B lymphocyte, lung Schwann, lung squamous, lung macrophage, lung osteoblast, neuroendocrine, lung alveolar, stomach epithelial, and stomach fibroblast.

As used herein, the term “tissue” is intended to mean a collection or aggregation of cells that act together to perform one or more specific functions in an organism. The cells can optionally be morphologically similar. Exemplary tissues include, but are not limited to, epididymidis, eye, muscle, skin, tendon, vein, artery, blood, heart, spleen, lymph node, bone, bone marrow, lung, bronchi, trachea, gut, small intestine, large intestine, colon, rectum, salivary gland, tongue, gall bladder, appendix, liver, pancreas, brain, stomach, skin, kidney, ureter, bladder, urethra, gonad, testicle, ovary, uterus, fallopian tube, thymus, pituitary, thyroid, adrenal, or parathyroid. Tissue can be derived from any of a variety of organs of a human or other organism. A tissue can be a healthy tissue or an unhealthy tissue. Examples of unhealthy tissues include, but are not limited to, malignancies in reproductive tissue, lung, breast, colorectum, prostate, nasopharynx, stomach, testes, skin, nervous system, bone, ovary, liver, hematologic tissues, pancreas, uterus, kidney, lymphoid tissues, etc. The malignancies may be of a variety of histological subtypes, for example, carcinoma, adenocarcinoma, sarcoma, fibroadenocarcinoma, neuroendocrine, or undifferentiated.

As used herein, the term “fixed cell” or “fixation” method refers to the aldehyde or alcohol based fixatives, such as paraformaldehyde, glutaraldehyde, methanol, and formalin, or combinations thereof that halt any biochemical reactions, and preserve the cell and/or tissue from decay due to autolysis or putrefaction.

As used herein, a “permeabilizing agent” removes the protective boundary of lipids often surrounding cellular macromolecules. Disruption of cellular lipid barriers via administration of a permeabilizing agent can provide enhanced physical access to cellular macromolecules, such as RNA, that might otherwise be relatively inaccessible. Examples of permeabilizing agents include, without limitation: Triton X-100, NP-40, methanol, acetone, Tween 20, and saponin.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIG. 1 shows a schematic of reagent loading in microwell arrays for single-cell sequencing in a Cas13-based molecular diagnostic application. Guide RNA molecules were loaded in droplets in microwells and lyophilized. Insets on the top two panels show the corresponding fluorescent images of guide RNAs. The bottom two panels show the successful reactivity of loaded guide RNA in microwells without well-to-well crosstalk, as demonstrated by the adjacent wells. Quantification of the fluorescent images indicated that the guide signal was observed only at the assay time point (3 hours) and only with the guide RNA matching the sample.

FIGS. 2A-2D show the instant disclosure method of binding a padlock probe and inducing rolling circle amplification (RCA) directly from an mRNA. FIG. 2A demonstrates that an inefficient reverse transcription step of extant rolling circle probe capture methods can be offset and/or ameliorated by hybridizing padlocks directly to the RNA transcript of interest using SplintR ligase to achieve padlock circularization. FIG. 2B shows that direct RNA detection and gel clearing substantially improved in situ detection efficiency in tissue and permitted in situ sequencing of endogenous transcripts. FIG. 2C shows quantification obtained from FIG. 2B. FIG. 2D shows direct RNA detection of B and T cell lineage markers, in this case in a solid tissue of fresh-frozen mouse spleen.

FIG. 3 demonstrates the experimental steps of single-cell combinatorial indexing on amplified RNAs. At top, padlock probes hybridize to RNA targets of interest in situ. Target sequences are then captured through reverse transcriptase gap fill and ligation. Next, circularized padlocks undergo rolling circle amplification (RCA) with products subsequently bound and extended by index adaptor containing primers. Amplicons containing the target sequence, UMIs and adaptors in the fixed cells then undergo combinatorial indexing to attach a cell-specific combination of bar codes. The final product then undergoes an indexed PCR, which is followed by sequencing (optionally next-generation sequencing).

FIG. 4 provides a flow chart of the single-cell combinatorial indexing of amplified RNAs disclosed herein for single-cell RNA sequencing. In situ padlock hybridization, reverse transcriptase gap fill, ligation, and RCA priming are shown in FIGS. 2A and 3, above. RCA, RCA product priming and extension are also shown in FIG. 3, above. Cell loading to the microwell array containing split and pool barcodes are shown in FIG. 3 above. Cell recovery from the microwell array was performed. Finally, the final PCR to produce an RNA-seq library for next generation sequencing is shown in FIG. 3 above.

FIG. 5 shows the relationship between the number of barcoding rounds, the number of microwells in which the cells are split, and the maximum number of cells per run. Notably, the relationship shows robust linear scaling. Accordingly, it is contemplated in certain embodiments of the instant disclosure that split-pool barcoding is performed between one and eight times (i.e., 1, 2, 3, 4, 5, 6, 7 and/or 8 times).

FIG. 6 shows a list of targeted cellular pathways and associated genes explicitly contemplated for the instant disclosure (list culled from U.S. Pat. No. 8,771,945).

### DETAILED DESCRIPTION OF THE INVENTION

The present disclosure is directed, at least in part, to the discovery that precisely quantitative single-cell nucleic acid sequencing (e.g., RNA sequencing, including mRNAs, snRNAs, lncRNAs, siRNAs, and gRNAs) can be obtained at scale from a tissue sample that has been treated with fixation and permeabilizing agents and subjected to padlock capture probes and rolling circle amplification (RCA). The disclosure allows for high accuracy, efficiency, specificity, and cell throughput of single-cell sequencing performed upon a tissue sample, cell sample, nuclei sample and/or extract or other sample. The improved accuracy and efficiency of single-cell sequencing disclosed herein enables the acquisition of single or combinatorial gene expression profiles associated with disease phenotypes, evaluation of nucleic acid therapy delivery and/or integration into tissues or cells, including, e.g., delivery of siRNAs, cellular CRISPR/Cas9 gRNAs, expression of CRISPR/Cas9 or TALEN plasmid(s), viral vectors (e.g., AAV), and expression of vectors/plasmids in general, among other applications. The instant disclosure also enables precise quantitative detection of snRNAs and lncRNAs, thereby allowing for the first time elucidation of a number of biological pathways that have been heretofore inaccessible.

It is further contemplated that quantitative detection of nucleic acid sequences as disclosed herein can enable improved measurement of, e.g., sequence barcodes, such as those used in the CITE-Seq process (Stoeckius et al. Nature Methods. 14: 865-868) and/or REAP-Seq process (Peterson et al. Nature Biotechnology. 35: 936-939), where quantitative measurement of nucleic acid barcodes is used as a proxy for antibody and/or antibody-bound protein levels. Such CITE-Seq and REAP-Seq processes are provided as examples among various other approaches where improved quantitative nucleic acid sequence measurement at low abundance and/or in single cells, such as that provided by certain aspects of the instant disclosure, is advantageous.

Certain aspects of the present disclosure therefore provide for improved quantitative detection of nucleic acid sequence barcodes, which in embodiments allows for highly sensitive quantitative detection of barcoded antibody levels and/or highly sensitive quantitative detection of barcoded antibody-bound protein levels (e.g., where specific antibodies are labeled with a barcoded oligonucleotide that is specific to each barcoded antibody's target protein—in such approaches, the oligonucleotide barcode can serve as a target nucleic acid sequence for the capture probes of the instant disclosure).

Various expressly contemplated components of certain compositions and methods of the instant disclosure are considered in additional detail below.

Single-cell (SC) molecular profiling methods have already made major impacts on biomedical research as such methods have recently transitioned into the mainstream, doing so alongside pre-existing SC-sensitive approaches like FACS. Breakthroughs and rapid progress have made SC resolution at many “omics” (ie. genomics, proteomics, transcriptomics, etc.) levels possible. While these techniques have helped to resolve cellular heterogeneity found within complex tissues, and without prior knowledge of cell states, costs have remained prohibitively high for most applications. This limitation has significantly hindered atlasing efforts (18), large clinical studies, discovery of rare subpopulations, and medium/large scale genetic screens with rich molecular readouts (19, 20).

Technical breakthroughs have driven performance and cost improvements of SC molecular profiling, and like next-generation sequencing (NGS) before it, SC analysis is now increasingly applied directly to patient care and pharmaceutical research. SC sequencing applications were critically limited by extant methods' pricing, of approximately $0.10/cell for sample preparation and $0.10/cell for sequence data generation when performed upon extant methods' highest-throughput platforms. Specifically, RNA processing of 100,000 cells to access 50 rare cells (0.05% abundance) via traditional methods has cost approximately $20,000, or $400 per cell of interest (21). Costs for experiments analyzing abundant cell types were similarly high. For example, a small case-control study with 10 subjects/group where 20,000 cells per sample were analyzed cost $80,000 per time point. A genome-wide PERTURB-seq screen of 80,000 CRISPR guide RNAs (˜4 per gene) replicated at 100 cells per guide (8,000,000 cells) was estimated to have cost approximately $1,600,000 under a single condition in a single cell type. There is therefore a need for major cost reductions in both sample preparation and sequencing cost for the field to realize the potential impact of SC sequencing.

The performance of available SC methods has been sorely in need of improvement. Known approaches for single-cell sequencing have been limited in accuracy and efficiency, at least in part by extant methods' reliance on probe hybridization as a proxy readout for an RNA sequence measurement. Because of the low accuracy, efficiency, and specificity of probe hybridization, known methods of single-cell sequencing yield outputs that are heavily biased towards higher frequency RNA transcripts. Reliance upon probe hybridization has also limited the magnitude of the cell throughput of single-cell sequencing. In particular, extant approaches have rendered only 5-15% of mRNA molecules detectable (22, 23). This sensitivity limit has heretofore been a major challenge to many SC sequencing applications because key lineage-defining regulatory genes like transcription factors have been undetectable in single cells due to their low expression levels (24) while poor sensitivity made gene-gene correlations difficult to detect (25). Available methods only detected mRNA sequence adjacent to the barcode, relying on polyadenylation, which heretofore has prevented isoform and small RNA analysis. Further, existing high-throughput transcriptome-wide methods unnecessarily required 50,000-500,000 reads per cell, with poor quantification of the 500-2000 genes escaping dropout. Many highly expressed genes were of little interest in studies, yet absorbed much of the sequencing effort. Additional dispersion in molecule-to-molecule PCR amplification of random length molecules exacerbated this effect. Unique molecular identifiers (UMIs) improved quantitation but not sequencing effort (26).

Prior to the instant disclosure, droplet technology has dominated SC sample preparation, wherein DNA barcode-bearing beads have been used to tag molecules from a given cell encapsulated in a droplet. The leading process is known as Sci-Seq, a similarly-performing approach which appends molecules from a permeabilized cell with a unique combination of barcode sequences in an iterative split and pool (S&P) approach (27-29). The Sci-seq approach requires no specialized microfluidics or bead reagents. However, the S&P process employed with the Sci-seq approach required that a single sample of cells was broken up into many separate reactions, in thousands of manual or robotic liquid handling operations, involving milliliters of reagents, and was thus susceptible to sensitivity limits due to cellular and molecular dropout. The basic consumables and capital equipment (automation) costs of Sci-seq have also put a significant floor on the cost per cell of using a conventional S&P approach, and was estimated to be within a factor of two of the cost of inputs for droplet approaches. Furthermore, all available high-throughput SC sequencing approaches were non-integrated, utilizing multiple pieces of equipment and significant hands-on time, limiting throughput and cost performance.

The instant disclosure describes herein a microfluidic implementation of split and pool SC sample preparation that provides a major advance in cost versus existing droplet and S&P approaches by addressing all three known cost drivers for sequence sample preparation: consumables (addressed herein via miniaturization and consumable-free dispensing), capital equipment (addressed herein via replacement of >$100 k robots with simple, low-cost consumables), and labor (addressed herein via process integration that reduces hands-on time). In contrast, previous variations of S&P labeling worked around the pricing of commercial systems but did not impact cost in a fundamental way (30, 31). In addition, in some embodiments the instant disclosure describes an improved library construction approach called SCIARseq (Single-cell Combinatorial Indexing of Amplified RNAs) that reduces sequencing costs while improving the technical performance and biological reach of SC genomics. Development and automation of this approach can decrease costs per cell >1000 fold, increase scale >100 fold, and also drastically reduce user handling time. The advances described herein therefore provide the field with a process for regularly executing very large-scale atlasing projects, clinical studies, and single-cell screens.

Sensitivity of detection for low-abundance nucleic acid sequences is one of the significant advantages of the methods disclosed herein. In particular, the methods disclosed herein are estimated as capable of detecting as little as a single copy of a nucleic acid sequence (e.g., a transcript) per cell (akin to the levels of sensitivity of transcript measurement recently described for the BOLORAMIS approach of Iyer et al. BioRxiv doi.org/10.1101/281121).

### Amplification Methods

A method as set forth herein can employ any of a variety of amplification techniques. Exemplary amplification techniques that can be used include, but are not limited to, polymerase chain reaction (PCR), rolling circle amplification (RCA), multiple displacement amplification (MDA), and random prime amplification (RPA).

RCA techniques can be modified for use in a method of the present disclosure. Exemplary components that can be used in an RCA reaction and principles by which RCA produces amplicons are described, for example, in Lizardi et al., Nat. Genet. 19:225-232 (1998) and U.S. Patent Publication No. 2007/0099208, each of which is incorporated herein by reference. The primers can be one or more of the universal primers described herein.

MDA techniques can be modified for use in a method of the present disclosure. Some basic principles and useful conditions for MDA are described, for example, in Dean et al., Proc Natl. Acad. Sci. USA 99:5261-66 (2002); Lage et al., Genome Research 13:294-307 (2003); Walker et al., Molecular Methods for Virus Detection, Academic Press, Inc., 1995; Walker et al., Nucl. Acids Res. 20:1691-96 (1992); U.S. Pat. Nos. 5,455,166; 5,130,238; and 6,214,587, each of which is incorporated herein by reference.

In particular embodiments, a combination of the above-exemplified amplification techniques can be used. For example, RCA and MDA can be used in a combination wherein RCA is used to generate a concatameric amplicon in solution (e.g. using solution-phase primers). The amplicon can then be used as a template for MDA using primers, optionally that are attached to a bead or other solid support (e.g. universal primers).

In some embodiments, a permeabilized padlock probe is used in combination with the rolling circle amplification (RCA) method to amplify an RNA target sequence. In some embodiments, combinatorial indexing of barcode sequences is further applied to cells to identify the cell of origin of the RNA target sequence.

Nucleic acid probes that are used in a method set forth herein or present in an apparatus or composition of the present disclosure can include barcode sequences, and for embodiments that include a plurality of different nucleic acid probes, each of the probes can include a different barcode sequence from other probes in the plurality. Barcode sequences can be any of a variety of lengths.

Longer sequences can generally accommodate a larger number and variety of barcodes for a population. Generally, all probes in a plurality will have the same length barcode (albeit with different sequences), but it is also possible to use different length barcodes for different probes. A barcode sequence can be at least 2, 4, 6, 8, 10, 12, 15, 20 or more nucleotides in length. Alternatively or additionally, the length of the barcode sequence can be at most 20, 15, 12, 10, 8, 6, 4 or fewer nucleotides. Examples of barcode sequences that can be used are set forth, for example, in U.S. Patent Publication No. 2014/0342921 and U.S. Pat. No. 8,460,865, each of which is incorporated herein by reference.

Exemplary nucleic acid detection methods include, but are not limited to, nucleic acid sequencing of a probe, hybridization of nucleic acids to a probe, ligation of nucleic acids that are hybridized to a probe, extension of nucleic acids that are hybridized to a probe, extension of a first nucleic acid that is hybridized to a probe followed by ligation of the extended nucleic acid to a second nucleic acid that is hybridized to the probe, or other methods known in the art such as those set forth in U.S. Pat. No. 8,288,103 or 8,486,625, each of which is incorporated herein by reference.

Various combinations of these states and stages can be used to expand the number of barcodes that can be decoded well beyond the number of distinct labels available for decoding. Such combinatorial methods are set forth in further detail in U.S. Pat. No. 8,460,865 or Gunderson et al., Genome Research 14:870-877 (2004), each of which is incorporated herein by reference.

It is contemplated that certain oligonucleotides of the instant disclosure can also include a linker (optionally a cleavable linker); a Unique Molecular Identifier (UMI) which differs for each priming site (as described below and as known in the art, e.g., see WO 2016/040476); a spatial barcode as described above and elsewhere herein; and a common sequence (“PCR handle”) to enable PCR amplification.

Exemplary split-and-pool synthesis of the barcode: to generate the cell barcode, the pool is repeatedly split into four equally sized oligonucleotide synthesis reactions, to which one of the four DNA bases is added, and then pooled together after each cycle, in a total of 12 split-pool cycles. The barcode synthesized reflects that unique (or sufficiently unique) path through the series of synthesis reactions. The result is a pool of barcodes, each possessing one of 4¹² (16,777,216) possible sequences on its entire complement of primers. Extension of the split-pool process can provide production of an even greater number of possible spatial barcode sequences for use in the compositions and methods of the instant disclosure. However, as noted above, functional use of barcodes does not require complete non-redundancy of barcodes in an array. Rather, provided that the majority of such barcodes are unique to a cell within a microarray, it is expressly contemplated that an array possessing only a small fraction of (e.g., even up to 10%, 20%, 30% or 40% or more) non-unique spatial barcodes (e.g., attributable to an artifact such as non-randomness of cell association having occurred during pool-and-split rounds, or simply to the likelihood that an array of a million cells derived from a ten million-fold complex library would still be expected to include a number of cells having redundant spatial barcodes in pairwise comparisons) could still yield a high rate of cell identification, where removal or other adjustment (averaging or other such adjustment) of any cells that turn out to be redundant in barcode within the array could be simply performed, e.g., during post-sequencing analysis.

Exemplary synthesis of a unique molecular identifier (UMI). Following the completion of the “split-and-pool” synthesis cycles described above for generation of barcodes, eight rounds of degenerate synthesis with all four DNA bases available during each cycle, such that each individual primer receives one of 4¹² (1,677,7216) possible sequences (UMIs). A padlock probe comprising a UMI is thereby provided that allows distinguishing the RNA transcript of interest.

A nucleic acid probe used in a composition or method set forth herein can include a target capture moiety. In particular embodiments, the target capture moiety is a target capture sequence. The target capture sequence is generally complementary to a target sequence such that target capture occurs by formation of a probe-target hybrid complex. A target capture sequence can be any of a variety of lengths including, for example, lengths exemplified above in the context of barcode sequences.

Extension of probes can be carried out using methods exemplified herein or otherwise known in the art for amplification of nucleic acids or sequencing of nucleic acids. In particular embodiments one or more nucleotides can be added to the 3′ end of a nucleic acid, for example, via polymerase catalysis (e.g. DNA polymerase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g. ligase catalysis) methods. A nucleic acid can be extended in a template directed manner, whereby the product of extension is complementary to a template nucleic acid that is hybridized to the nucleic acid that is extended. Exemplary methods for extending nucleic acids are set forth in US Pat. App. Publ. No. US 2005/0037393 or U.S. Pat. No. 8,288,103 or 8,486,625, each of which is incorporated herein by reference.

All or part of a target nucleic acid that is hybridized to a nucleic acid probe can be copied by extension. For example, an extended probe can include at least, 1, 2, 5, 10, 25, 50, 100, 200, 500, 1000 or more nucleotides that are copied from a target nucleic acid. The length of the extension product can be controlled, for example, using reversibly terminated nucleotides in the extension reaction and running a limited number of extension cycles. The cycles can be run as exemplified for SBS techniques and the use of labeled nucleotides is not necessary.

Accordingly, an extended probe produced in a method set forth herein can include no more than 1000, 500, 200, 100, 50, 25, 10, 5, 2 or 1 nucleotides that are copied from a target nucleic acid. Of course extended probes can be any length within or outside of the ranges set forth above.

### Tissue Samples and Sectioning

In some embodiments, a tissue section is employed. The tissue can be derived from a multicellular organism. Exemplary multicellular organisms include, but are not limited to a mammal, plant, algae, nematode, insect, fish, reptile, amphibian, fungi or Plasmodium falciparum. Exemplary species are set forth previously herein or known in the art. The tissue can be freshly excised from an organism or it may have been previously preserved for example by freezing, embedding in a material such as paraffin (e.g. formalin fixed paraffin embedded samples), formalin fixation, infiltration, dehydration or the like. Optionally, a tissue section can be cryosectioned, using techniques and compositions as described herein and as known in the art. As a further option, a tissue can be permeabilized and the cells of the tissue lysed. Any of a variety of art-recognized lysis treatments can be used. Target nucleic acids that are released from a tissue that is permeabilized can be captured by nucleic acid probes, as described herein and as known in the art.

A tissue can be prepared in any convenient or desired way for its use in a method, composition or apparatus herein. Fresh, frozen, fixed or unfixed tissues can be used. A tissue can be fixed or embedded using methods described herein or known in the art.

A tissue sample for use herein, can be fixed by deep freezing at temperature suitable to maintain or preserve the integrity of the tissue structure, e.g. less than −20° C. In another example, a tissue can be prepared using formalin-fixation and paraffin embedding (FFPE) methods which are known in the art. Other fixatives and/or embedding materials can be used as desired. A fixed or embedded tissue sample can be sectioned, i.e. thinly sliced, using known methods. For example, a tissue sample can be sectioned using a chilled microtome or cryostat, set at a temperature suitable to maintain both the structural integrity of the tissue sample and the chemical properties of the nucleic acids in the sample. Exemplary additional fixatives that are expressly contemplated include alcohol fixation (e.g., methanol fixation, ethanol fixation), glutaraldehyde fixation and paraformaldehyde fixation.

### Permeabilizing Agents

Certain aspects of the instant disclosure feature permeabilizing agents, examples of which tend to compromise and/or remove the protective boundary of lipids often surrounding cellular macromolecules. Disruption of cellular lipid barriers via administration of a permeabilizing agent can provide enhanced physical access to cellular macromolecules, such as DNA, that might otherwise be relatively inaccessible. Specifically contemplated examples of permeabilizing agents include, without limitation: Triton X-100, NP-40, methanol, acetone, Tween 20, and saponin. In certain embodiments, fixation is performed with paraformaldehyde, optionally 4% paraformaldehyde. Optionally, permeabilization is performed with <1% TritonX-100, optionally 0.2% TritonX-100.

A particularly relevant source for a tissue sample is a human being. The sample can be derived from an organ, including for example, an organ of the central nervous system such as brain, brainstem, cerebellum, spinal cord, cranial nerve, or spinal nerve; an organ of the musculoskeletal system such as muscle, bone, tendon or ligament; an organ of the digestive system such as salivary gland, pharynx, esophagus, stomach, small intestine, large intestine, liver, gallbladder or pancreas; an organ of the respiratory system such as larynx, trachea, bronchi, lungs or diaphragm; an organ of the urinary system such as kidney, ureter, bladder or urethra; a reproductive organ such as ovary, fallopian tube, uterus, vagina, placenta, testicle, epididymis, vas deferens, seminal vesicle, prostate, penis or scrotum; an organ of the endocrine system such as pituitary gland, pineal gland, thyroid gland, parathyroid gland, or adrenal gland; an organ of the circulatory system such as heart, artery, vein or capillary; an organ of the lymphatic system such as lymphatic vessel, lymph node, bone marrow, thymus or spleen; a sensory organ such as eye, ear, nose, or tongue; or an organ of the integument such as skin, subcutaneous tissue or mammary gland. In some embodiments, a tissue sample is obtained from a bodily fluid or excreta such as blood, lymph, tears, sweat, saliva, semen, vaginal secretion, ear wax, fecal matter or urine.

A sample from a human can be considered (or suspected) healthy or diseased when used. In some cases, two samples can be used: a first being considered diseased and a second being considered as healthy (e.g. for use as a healthy control). Any of a variety of conditions can be evaluated, including but not limited to, cancer, an autoimmune disease, cystic fibrosis, aneuploidy, pathogenic infection, psychological condition, hepatitis, diabetes, sexually transmitted disease, heart disease, stroke, cardiovascular disease, multiple sclerosis or muscular dystrophy. Certain contemplated conditions include genetic conditions or conditions associated with pathogens having identifiable DNA abundance signatures.

### Low Prevalence RNA Transcripts

In some embodiments, the instant disclosure describes high throughput and sufficiently sensitive methods of single-cell RNA sequence amplification, indexing, and sequencing wherein lower prevalence RNA sequences are readily captured. In some embodiments, “lower prevalence RNA sequences” and/or “lower abundance RNA sequences” refer to the bulk of the genome, due to the bias towards higher prevalence “housekeeping” transcripts in traditional single-cell sequencing methods. Therefore, certain embodiments of the instant disclosure describe methods for capturing the sequence of most mRNAs by percentage of the genome, for small nuclear RNAs (snRNAs), for long non-coding RNAs (lncRNAs), for short-interfering RNAs, and for guide RNAs (gRNAs).

snRNAs are a class of small RNA molecule found within the splicing speckles of Cajal bodies of the nucleus. The length of an average snRNA is approximately 150 nucleic acids. They are transcribed by either RNA polymerase II or III. snRNAs are always in complex with small nuclear ribonucleoproteins and are involved in a number of disease pathologies, including but not limited to Spinal muscular atrophy, Dyskeratosis congenital, Prader-Willi syndrome, and Medulloblastoma. lncRNAs are RNA transcripts longer than 200 nucleotides that are not translated into protein. lncRNAs have been shown to regulate for example, but not limited to: gene transcription, post-transcriptional regulation such as splicing and translation, epigenetic regulation and X-chromosome regulation. The instant disclosure provides methods for high throughput and accurate measurement of snRNA and lncRNA sequences, making a significant contribution to the field's research and disease therapies.

siRNAs are a class of double-stranded RNA non-coding RNA molecules, 20-25 base pairs in length, similar to miRNA, and operating within the RNA interference (RNAi) pathway. siRNAs are also widely applied in exogenous methods of gene silencing for disease therapy and research applications. The instant disclosure provides methods for high throughput and accurate measurement of siRNA sequences, which will enhance the understanding of endogenous siRNAs species. The instant disclosure provides methods for high throughput and accurate measurement of siRNA sequences, which makes a significant contribution to the post-treatment verification of siRNA delivery to tissue in disease therapies.

The terms “guide RNA” and “gRNA” are used in DNA editing involving CRISPR and Cas9. For this prokaryote-originated DNA-editing system, the gRNA confers target sequence specificity to the CRISPR-Cas9 system. These gRNAs are non-coding short RNA sequences which bind to the complementary target DNA sequences. Guide RNA first binds to the Cas9 enzyme and the gRNA sequence guides the complex via pairing to a specific location on the DNA, where Cas9 performs its endonuclease activity by cutting the target DNA strand. In addition to expression of the Cas9 nuclease, the CRISPR-Cas9 system requires a specific RNA molecule to recruit and direct the nuclease activity to the region of interest. These guide RNAs take one of two forms: (1) a synthetic trans-activating CRISPR RNA (tracrRNA) plus a synthetic CRISPR RNA (crRNA) designed to cleave the gene target site of interest; and (2) a synthetic or expressed single guide RNA (sgRNA) that consists of both the crRNA and tracrRNA as a single construct. The crRNA and the tracrRNA form a complex which acts as the guide RNA for the Cas9 enzyme. The scaffolding ability of tracrRNA along with crRNA specificity can be combined into a single synthetic gRNA which simplifies guiding of gene alterations to a one component system which may increase efficiencies. The instant disclosure provides methods for high throughput and accurate measurement of these gRNAs, which makes a significant contribution to the post-treatment verification of CRISPR-Cas9 delivery to tissue in research and disease therapies.

### Cell Throughput Magnitude

Traditional methods of single-cell sequencing have rendered only 5-15% of mRNA molecules detectable (22, 23). The instant disclosure describes methods for obtaining more accurate sequencing data per cell, thereby reducing the number of cells needed to support a given analysis. Therefore, the instant disclosure increases the ratio of meaningful data to cells, i.e. it increases the number of cells which can be successfully processed for data from a given experimental run. In some embodiments, the instant disclosure describes processing of from 1×10⁶-1×10¹² cells per run, an improvement of up to 7× or more the magnitude of extant methods (21).

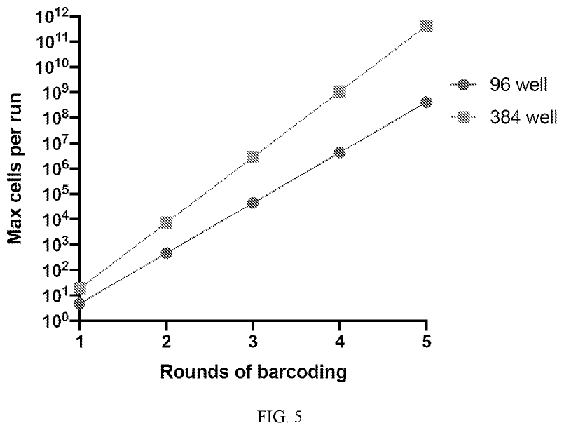

In some embodiments the number of barcoding rounds and microwells influences the maximum number of cells distinguishable per run, wherein the number of barcoding rounds scales linearly with the maximum number of cells distinguishable per run (see FIG. 5). In these embodiments, the length of the barcode is sufficient such that after combinatorial indexing, each cell will have an identifying sequence.

### Genes of Interest

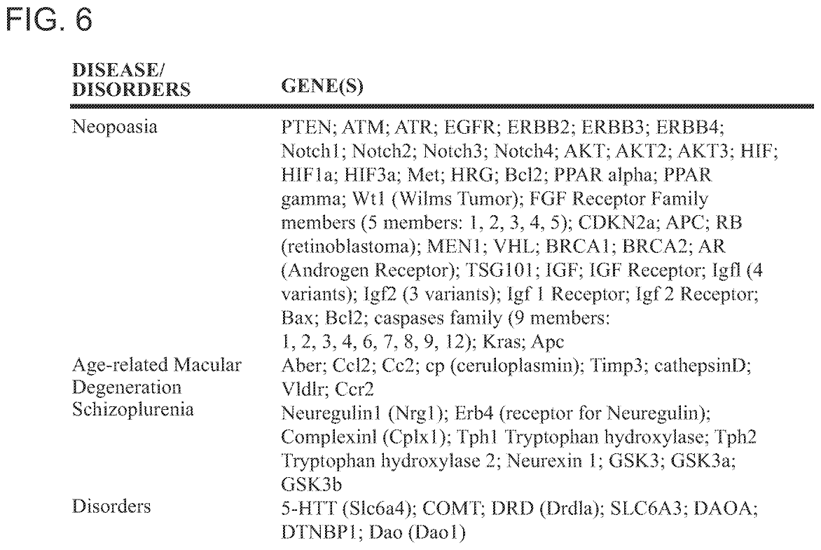

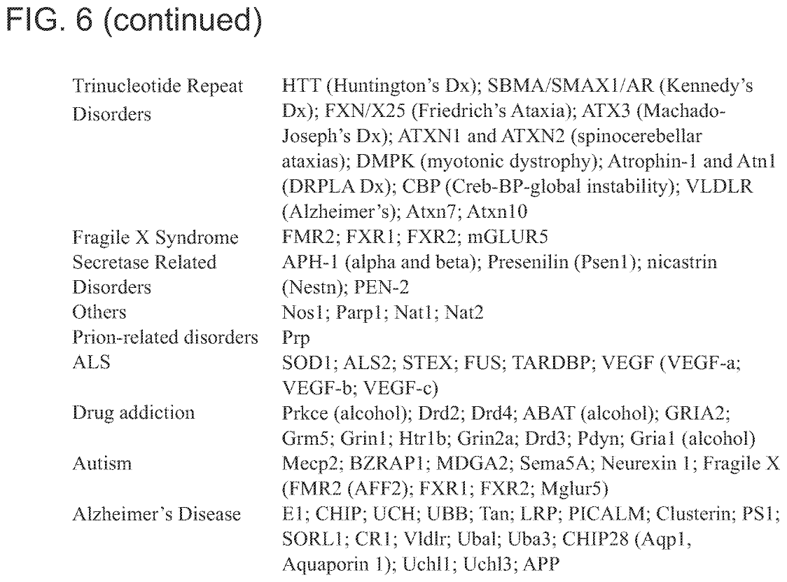

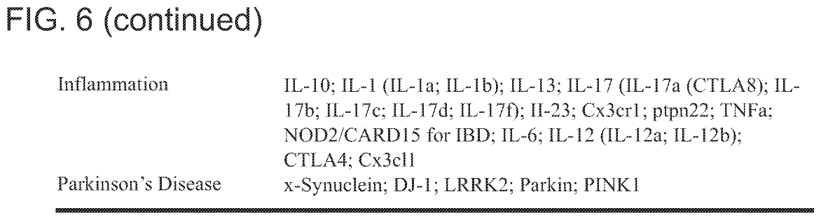

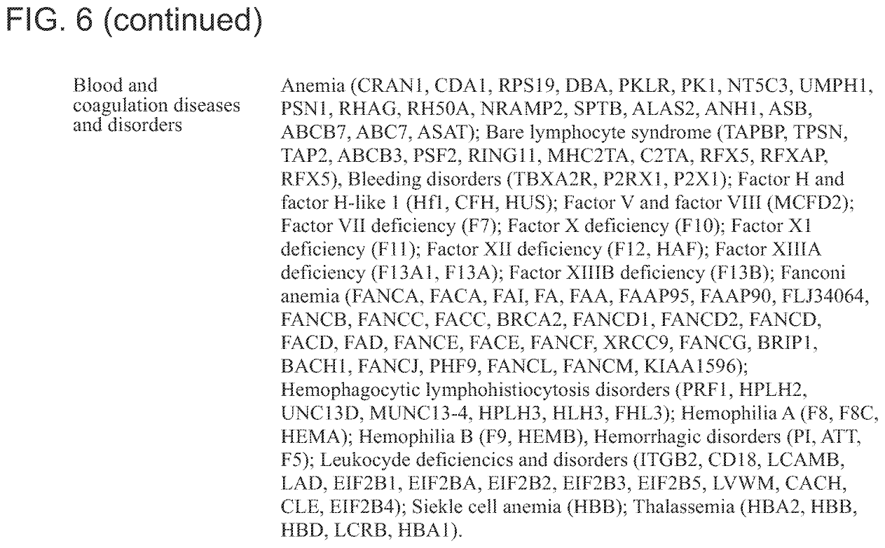

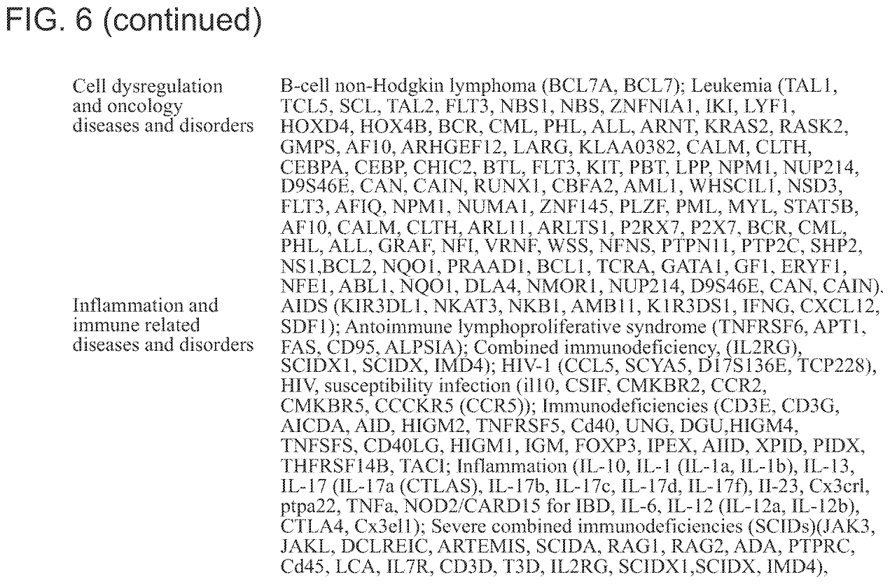

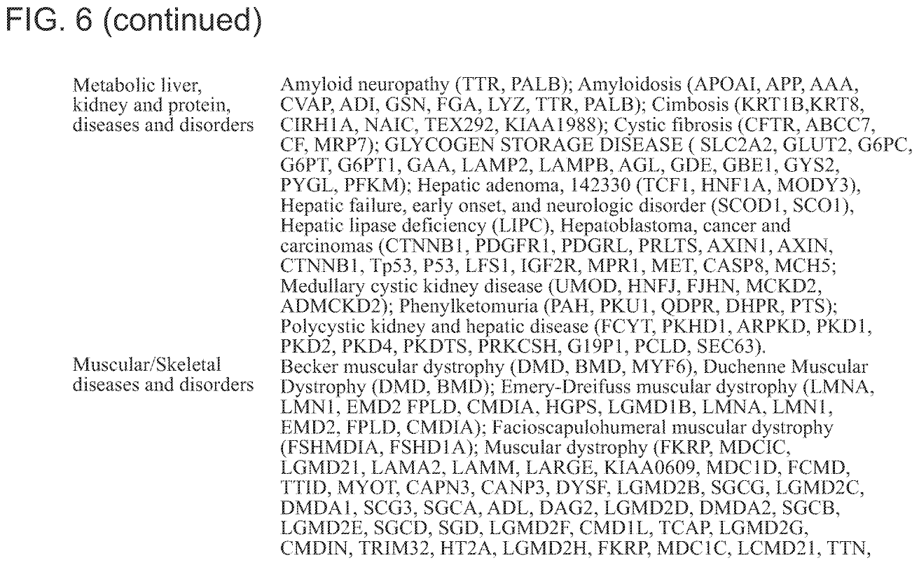

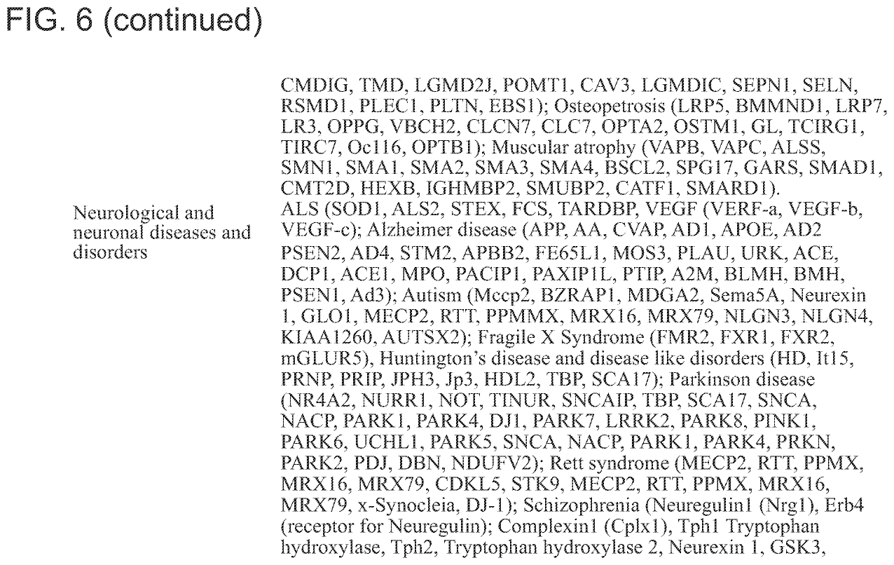

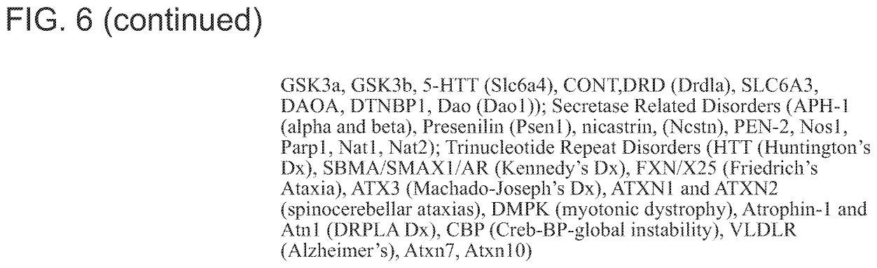

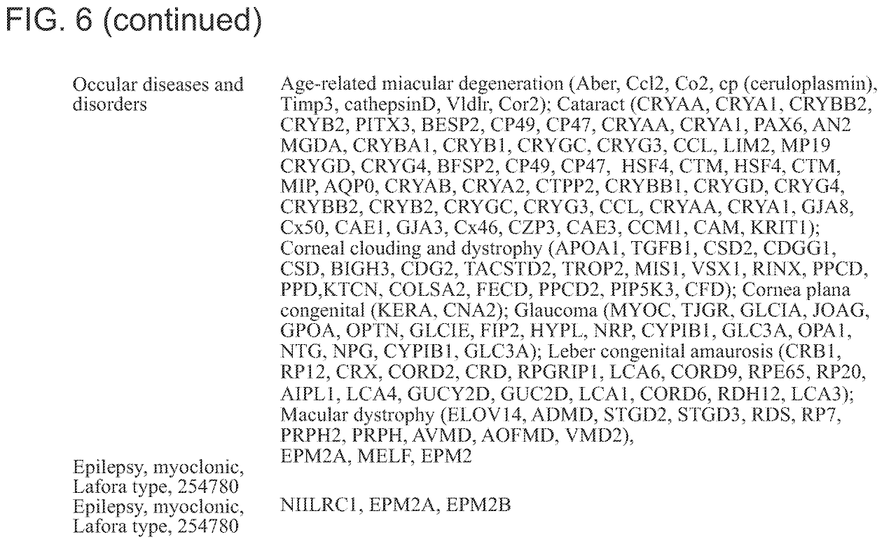

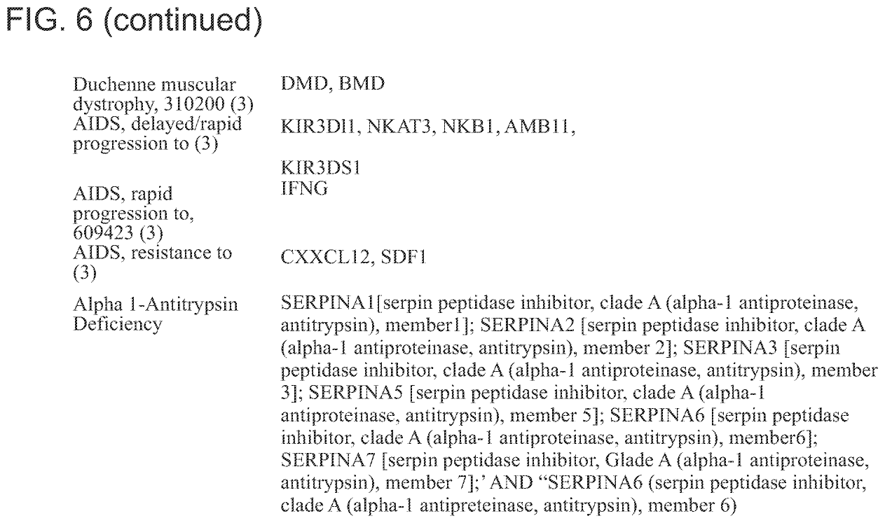

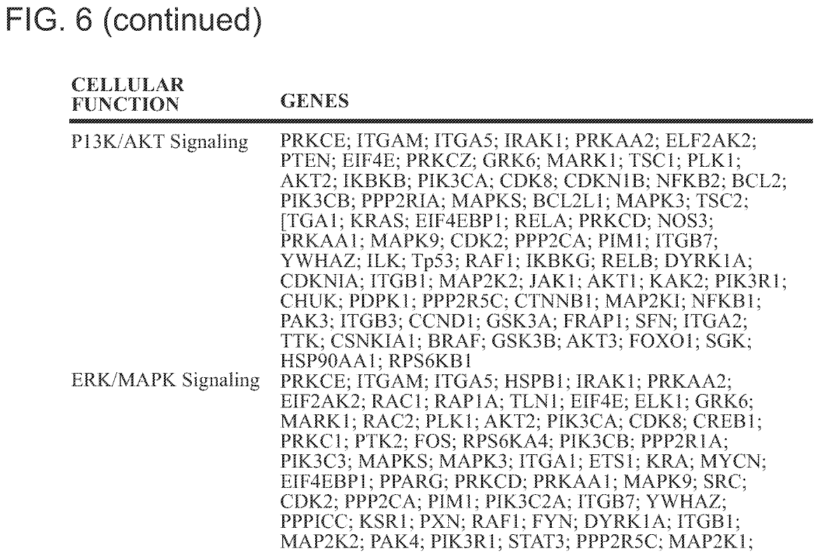

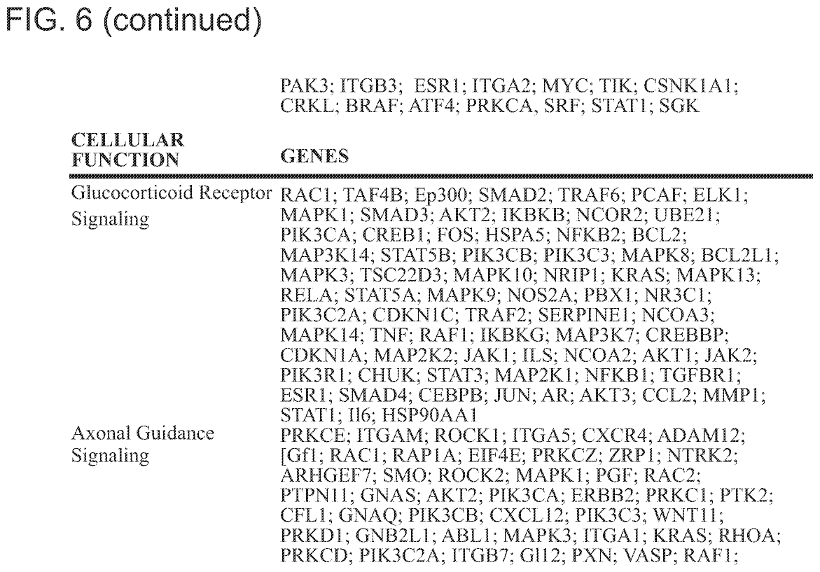

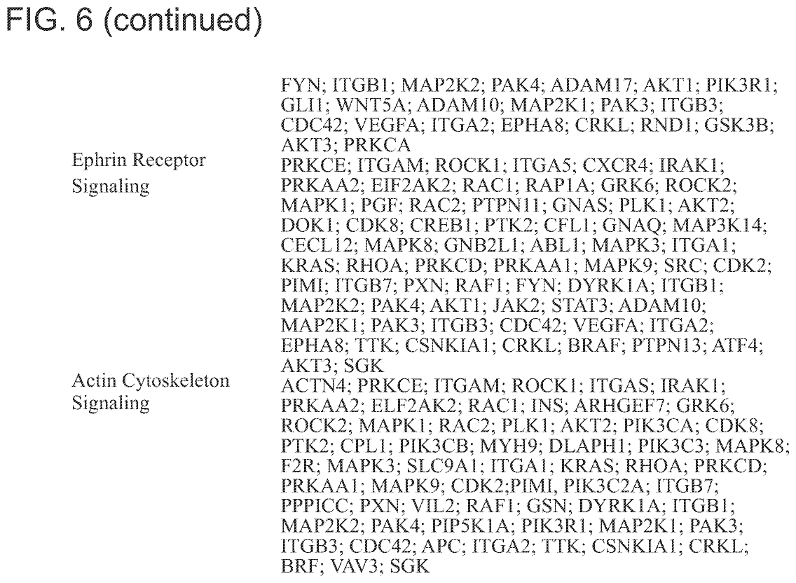

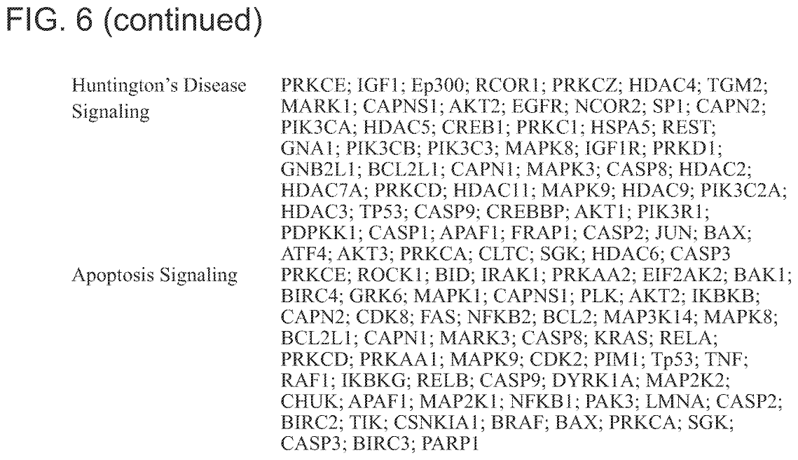

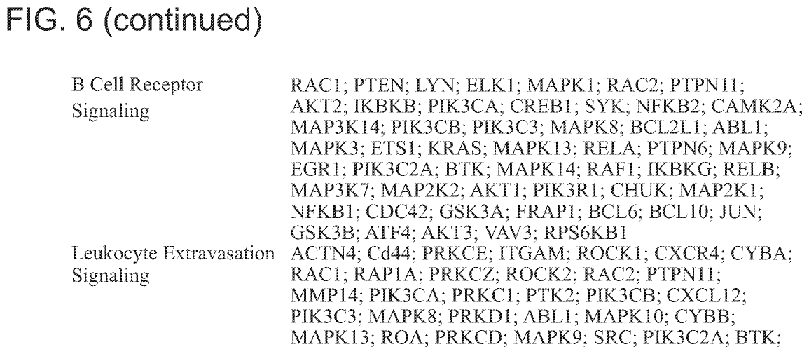

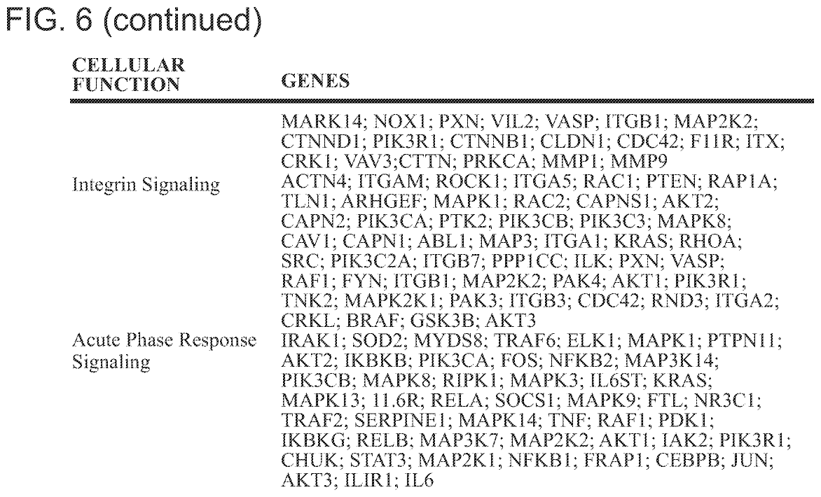

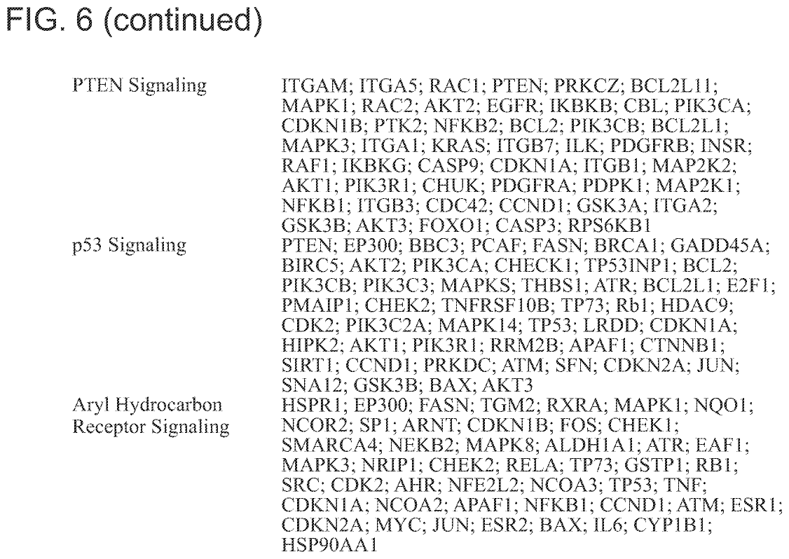

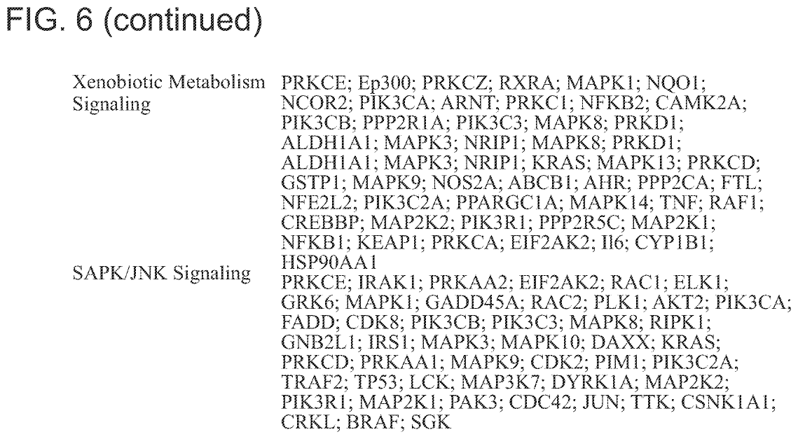

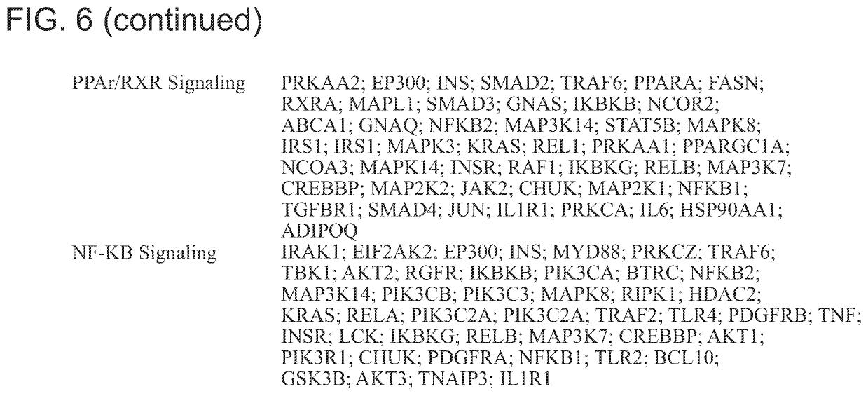

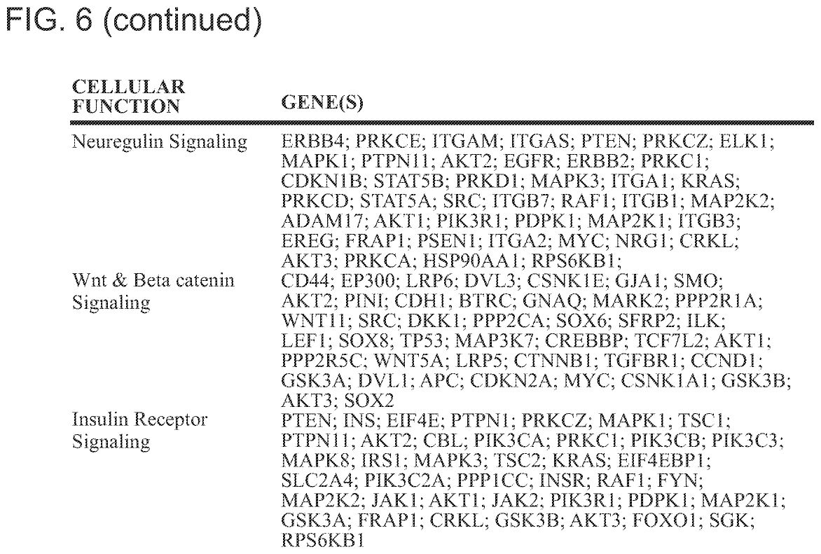

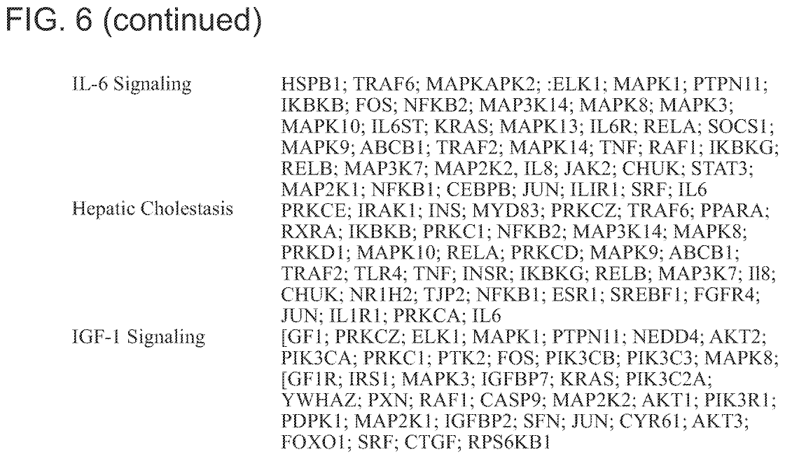

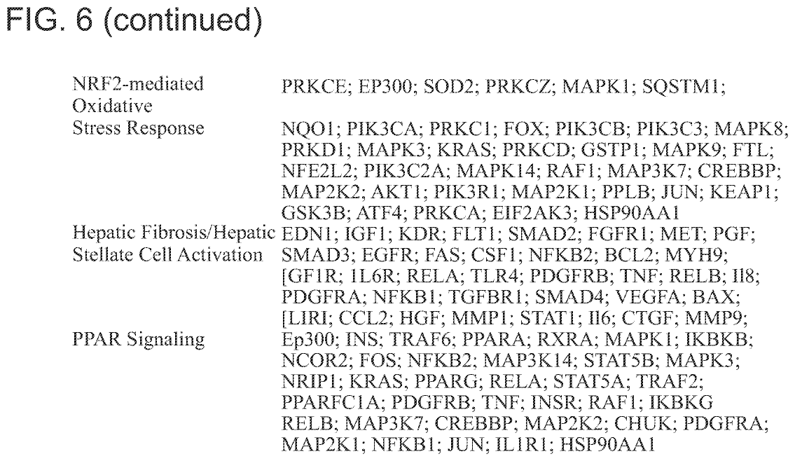

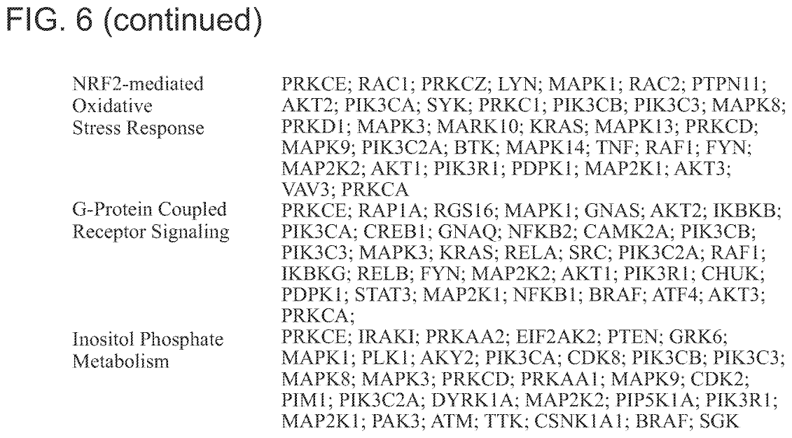

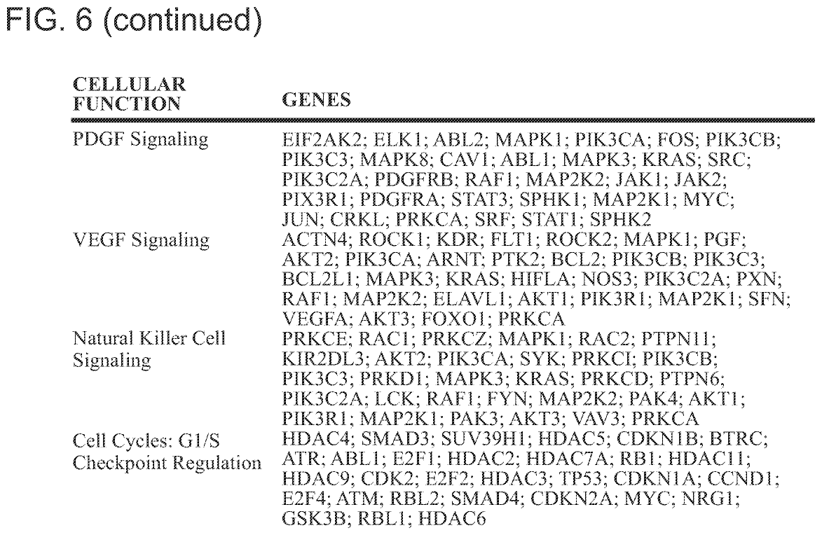

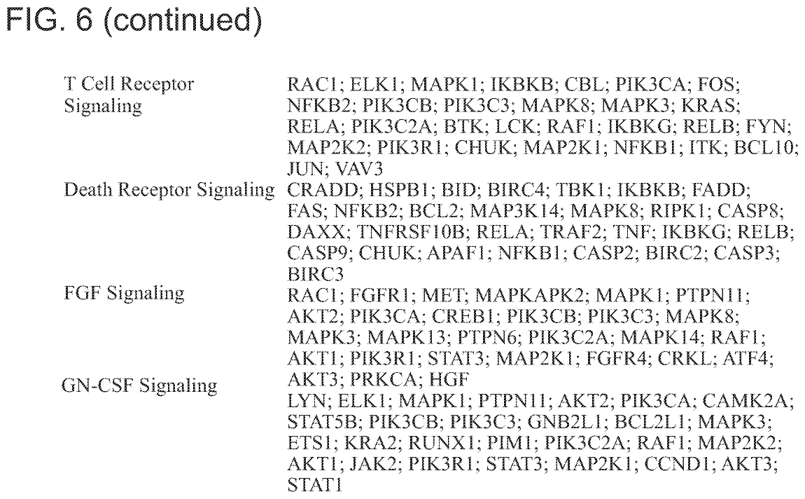

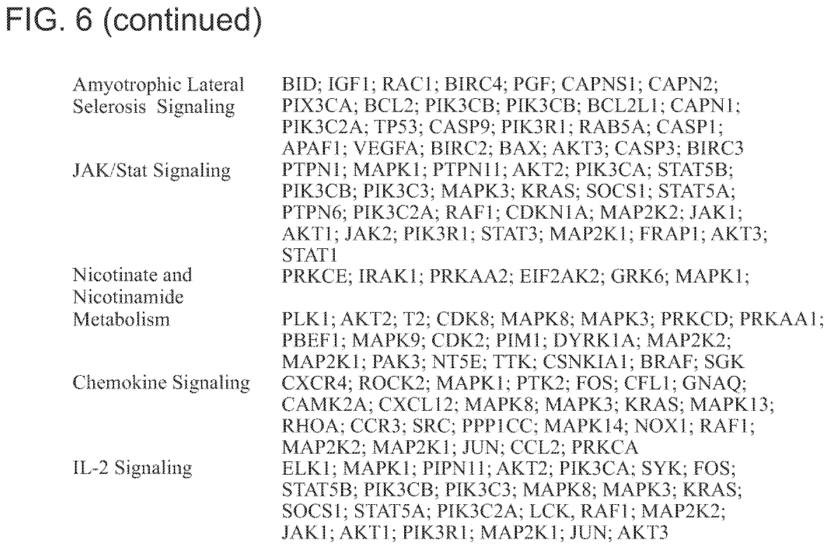

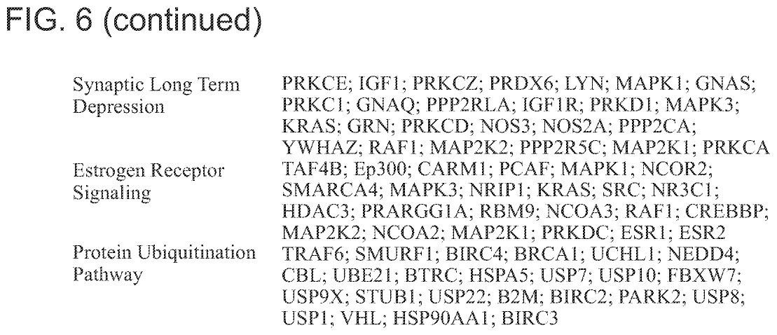

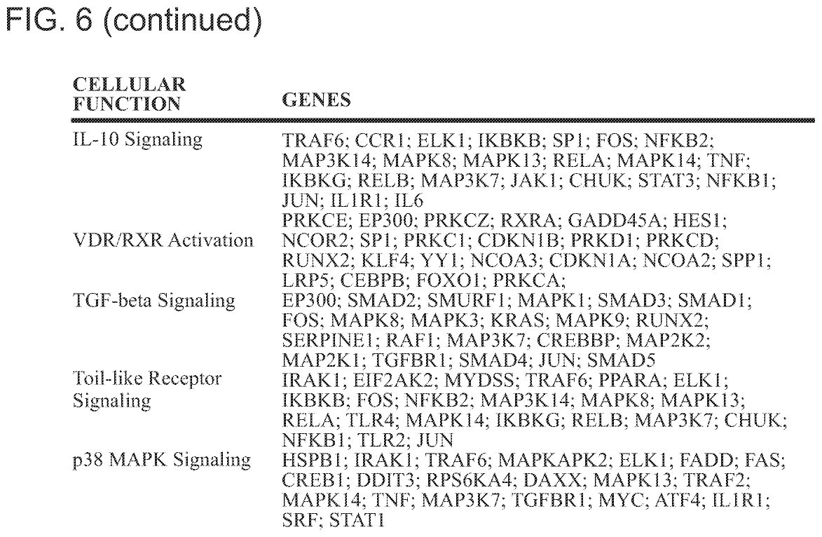

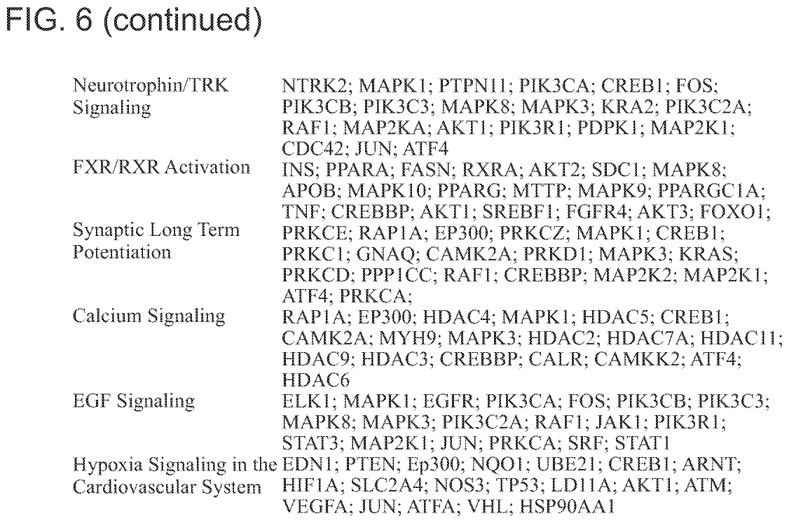

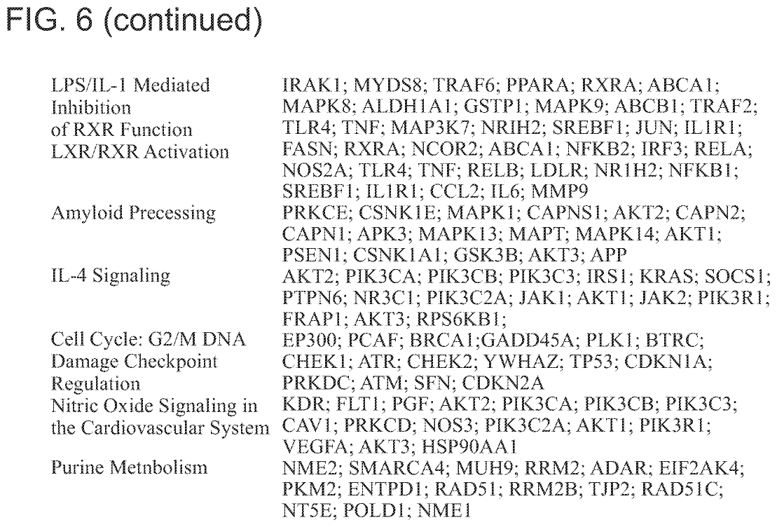

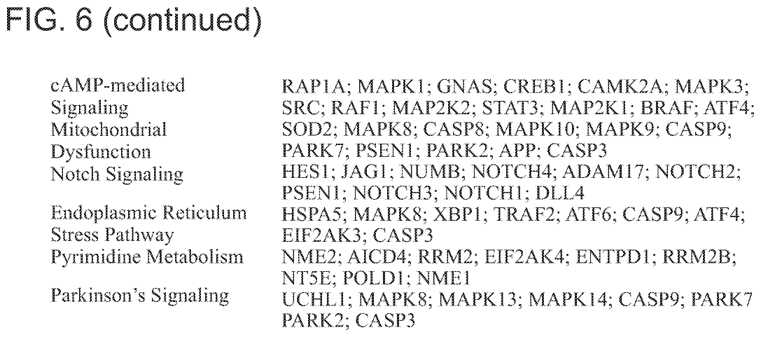

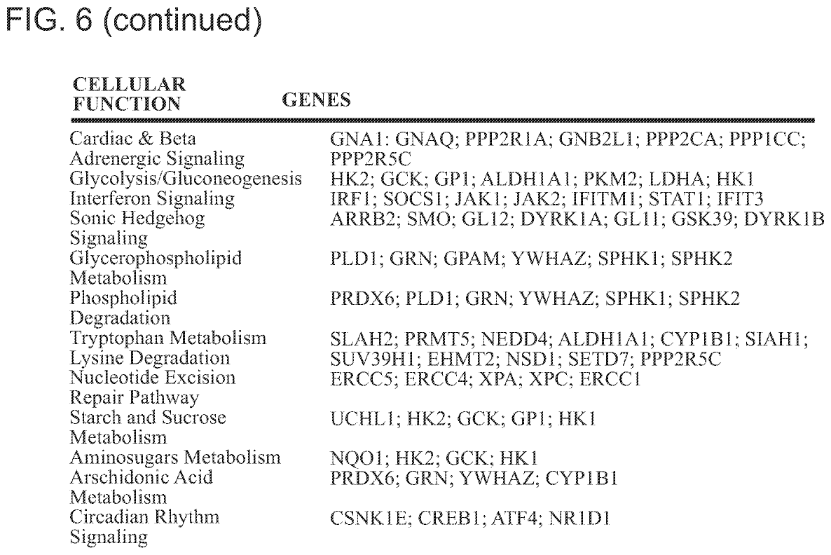

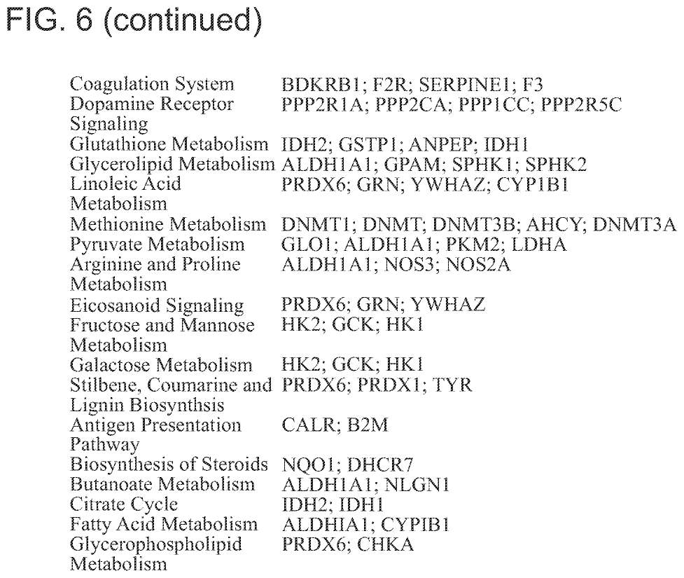

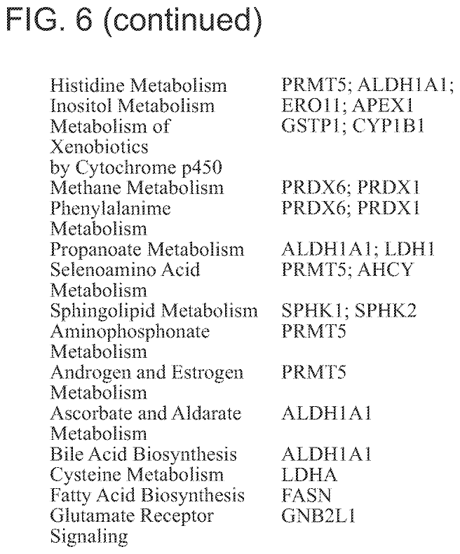

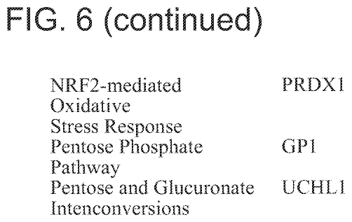

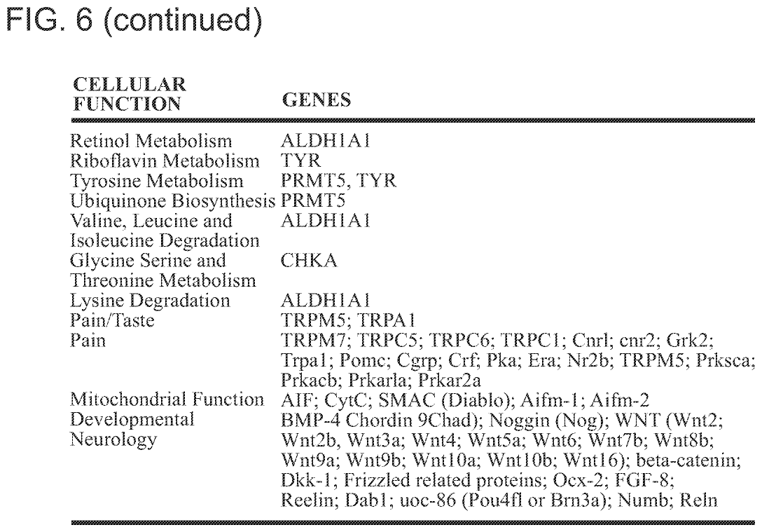

FIG. 6 provides exemplary pathways and associated genes/target nucleic acid sequences expressly contemplated by the current disclosure, without limitation.

### FIG. 6

### Sequencing Methods

Sequencing techniques, such as sequencing-by-synthesis (SBS) techniques, are a useful method for determining barcode sequences. SBS can be carried out as follows. To initiate a first SBS cycle, one or more labeled nucleotides, DNA polymerase, SBS primers etc., can be contacted with one or more features, optionally on a bead or other solid support (e.g. feature(s) where nucleic acid probes are attached to the bead or other solid support). Those features where SBS primer extension causes a labeled nucleotide to be incorporated can be detected. Optionally, the nucleotides can include a reversible termination moiety that terminates further primer extension once a nucleotide has been added to the SBS primer. For example, a nucleotide analog having a reversible terminator moiety can be added to a primer such that subsequent extension cannot occur until a deblocking agent is delivered to remove the moiety. Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with a composition, apparatus or method of the present disclosure are described, for example, in Bentley et al., Nature 456:53-59 (2008), PCT Publ. Nos. WO 91/06678, WO 04/018497 or WO 07/123744; U.S. Pat. Nos. 7,057,026, 7,329,492, 7,211,414, 7,315,019 or 7,405,281, and U.S. Patent Publication No. 2008/0108082, each of which is incorporated herein by reference.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11 (1), 3-11 (2001); Ronaghi et al. Science 281 (5375), 363 (1998); or U.S. Pat. Nos. 6,210,891, 6,258,568 or 6,274,320, each of which is incorporated herein by reference). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system.

Excitation radiation sources used for fluorescence based detection systems are not necessary for pyrosequencing procedures. Useful fluidic systems, detectors and procedures that can be used for application of pyrosequencing to apparatus, compositions or methods of the present disclosure are described, for example, in PCT Patent Publication No. WO2012/058096, US Patent Publication No. 2005/0191698 A1, or U.S. Pat. No. 7,595,883 or 7,244,559, each of which is incorporated herein by reference.

Sequencing-by-ligation reactions are also useful including, for example, those described in Shendure et al. Science 309:1728-1732 (2005); or U.S. Pat. No. 5,599,675 or 5,750,341, each of which is incorporated herein by reference. Some embodiments can include sequencing-by-hybridization procedures as described, for example, in Bains et al., Journal of Theoretical Biology 135(3), 303-7 (1988); Drmanac et al., Nature Biotechnology 16, 54-58 (1998); Fodor et al., Science 251 (4995), 767-773 (1995); or PCT Publication No. WO 1989/10977, each of which is incorporated herein by reference. In both sequencing-by-ligation and sequencing-by-hybridization procedures, target nucleic acids (or amplicons thereof) that are present at sites of an array are subjected to repeated cycles of oligonucleotide delivery and detection. Compositions, apparatus or methods set forth herein or in references cited herein can be readily adapted for sequencing-by-ligation or sequencing-by-hybridization procedures. Typically, the oligonucleotides are fluorescently labeled and can be detected using fluorescence detectors similar to those described with regard to SBS procedures herein or in references cited herein.

Some sequencing embodiments can utilize methods involving the real-time monitoring of DNA polymerase activity. For example, nucleotide incorporations can be detected through fluorescence resonance energy transfer (FRET) interactions between a fluorophore-bearing polymerase and 7-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); and Korlach et al. Proc. Natl. Acad. Sci. USA 105, 1 176-1 181 (2008), each of which is incorporated herein by reference.

Some sequencing embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies and Thermo Fisher subsidiary) or sequencing methods and systems described in U.S. Patent Publication Nos. 2009/0026082 A1; 2009/0127589 A1; 2010/0137143 A1; or U.S. Publication No. 2010/0282617 A1, each of which is incorporated herein by reference.

Nucleic acid hybridization techniques are also useful methods for determining barcode sequences. In some cases combinatorial hybridization methods can be used, see, e.g., U.S. Pat. No. 8,460,865, which is incorporated herein by reference. Such methods utilize labelled nucleic acid decoder probes that are complementary to at least a portion of a barcode sequence. A hybridization reaction can be carried out using decoder probes having known labels such that the location where the labels end up on, in some embodiments in a microwell or solid support identifies the nucleic acid probes according to rules of nucleic acid complementarity. In some cases, pools of many different probes with distinguishable labels are used, thereby allowing a multiplex decoding operation. The number of different barcodes determined in a decoding operation can exceed the number of labels used for the decoding operation. For example, decoding can be carried out in several stages where each stage constitutes hybridization with a different pool of decoder probes. The same decoder probes can be present in different pools but the label that is present on each decoder probe can differ from pool to pool (i.e. each decoder probe is in a different “state” when in different pools).

Some of the methods and compositions provided herein employ methods of sequencing nucleic acids. A number of DNA sequencing techniques are known in the art, including fluorescence-based sequencing methodologies (See, e.g., Birren et al, Genome Analysis Analyzing DNA, 1, Cold Spring Harbor, N.Y., which is incorporated herein by reference in its entirety). In some embodiments, automated sequencing techniques understood in that art are utilized. In some embodiments, parallel sequencing of partitioned amplicons can be utilized (PCT Publication No WO2006084132, which is incorporated herein by reference in its entirety). In some embodiments, DNA sequencing is achieved by parallel oligonucleotide extension (See, e.g., U.S. Pat. Nos. 5,750,341; 6,306,597, which are incorporated herein by reference in their entireties). Additional examples of sequencing techniques include the Church polony technology (Mitra et al, 2003, Analytical Biochemistry 320, 55-65; Shendure et al, 2005 Science 309, 1728-1732; U.S. Pat. Nos. 6,432,360, 6,485,944, 6,511,803, which are incorporated by reference), the 454 picotiter pyrosequencing technology (Margulies et al, 2005 Nature 437, 376-380; US 20050130173, which are incorporated herein by reference in their entireties), the Solexa single base addition technology (Bennett et al, 2005, Pharmacogenomics, 6, 373-382; U.S. Pat. Nos. 6,787,308; 6,833,246, which are incorporated herein by reference in their entireties), the Lynx massively parallel signature sequencing technology (Brenner et al. (2000). Nat. Biotechnol. 18:630-634; U.S. Pat. Nos. 5,695,934; 5,714,330, which are incorporated herein by reference in their entireties), and the Adessi PCR colony technology (Adessi et al. (2000). Nucleic Acid Res. 28, E87; WO 00018957, which are incorporated herein by reference in their entireties).

Next-generation sequencing (NGS) methods can be employed in certain aspects of the instant disclosure to obtain a high volume of sequence information in a highly efficient and cost effective manner. NGS methods share the common feature of massively parallel, high-throughput strategies, with the goal of lower costs in comparison to older sequencing methods (see, e.g., Voelkerding et al, Clinical Chem., 55: 641-658, 2009; MacLean et al, Nature Rev. Microbiol, 7-287-296; which are incorporated herein by reference in their entireties). NGS methods can be broadly divided into those that typically use template amplification and those that do not. Amplification-utilizing methods include pyrosequencing commercialized by Roche as the 454 technology platforms (e.g., GS 20 and GS FLX), the Solexa platform commercialized by Illumina, and the Supported Oligonucleotide Ligation and Detection (SOLiD™) platform commercialized by Applied Biosystems. Non-amplification approaches, also known as single-molecule sequencing, are exemplified by the HeliScope platform commercialized by Helicos Biosciences, SMRT sequencing commercialized by Pacific Biosciences, and emerging platforms marketed by VisiGen and Oxford Nanopore Technologies Ltd.

In pyrosequencing (U.S. Pat. Nos. 6,210,891; 6,258,568, which are incorporated herein by reference in their entireties), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al, Clinical Chem., 55-641-658, 2009; MacLean et al, Nature Rev. Microbiol, 7:287-296; U.S. Pat. Nos. 6,833,246; 7,115,400; 6,969,488, which are incorporated herein by reference in their entireties), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluorophore and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al, Clinical Chem., 55: 641-658, 2009; U.S. Pat. Nos. 5,912,148; and 6,130,073, which are incorporated herein by reference in their entireties) can initially involve fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing is employed (see, e.g., Astier et al, J. Am. Chem. Soc. 2006 Feb. 8; 128(5): 1705-10, which is incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore (or as individual nucleotides pass through the nanopore in the case of exonuclease-based techniques), this causes a change in the magnitude of the current through the nanopore that is distinct for each of the four bases, thereby allowing the sequence of the DNA molecule to be determined.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, which are incorporated herein by reference in their entireties). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is approximately 99.6% for 50 base reads, with approximately 100 Mb generated per run. The read-length is 100 base pairs. The accuracy for homopolymer repeats of 5 repeats in length is approximately 98%. The benefits of ion semiconductor sequencing are rapid sequencing speed and low upfront and operating costs.

In particular embodiments, a fluorescence microscope (e.g. a confocal fluorescent microscope) can be used to detect a biological specimen that is fluorescent, for example, by virtue of a fluorescent label. Fluorescent specimens can also be imaged using a nucleic acid sequencing device having optics for fluorescent detection such as a Genome Analyzer®, MiSeq®, NextSeq® or HiSeq® platform device commercialized by lllumina, Inc. (San Diego, Calif.); or a SOLiD™ sequencing platform commercialized by Life Technologies (Carlsbad, Calif.). Other imaging optics that can be used include those that are found in the detection devices described in Bentley et al., Nature 456:53-59 (2008), PCT Publ. Nos. WO 91/06678, WO 04/018497 or WO 07/123744; U.S. Pat. Nos. 7,057,026, 7,329,492, 7,211,414, 7,315,019 or 7,405,281, and US Pat. App. Publ. No. 2008/0108082, each of which is incorporated herein by reference.

An image of a biological specimen can be obtained at a desired resolution, for example, to distinguish tissues, cells or subcellular components. Accordingly, the resolution can be sufficient to distinguish components of a biological specimen that are separated by at least 0.5 μm, 1 μm, 5 μm, 10 μm, 50 μm, 100 μm, 500 μm, 1 mm or more. Alternatively or additionally, the resolution can be set to distinguish components of a biological specimen that are separated by at least 1 mm, 500 μm, 100 μm, 50 μm, 10 μm, 5 μm, 1 μm, 0.5 μm or less.

Kits The instant disclosure also provides kits containing agents of this disclosure for use in the methods of the present disclosure. Kits of the instant disclosure may include one or more containers comprising an agent and/or composition of this disclosure. In some embodiments, the kits further include instructions for use in accordance with the methods of this disclosure. In some embodiments, these instructions comprise a description of administration of the agent to diagnose, e.g., a disease and/or malignancy.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. The container may further comprise a pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Low-Cost SC Sample Preparation by Microfluidic Split/Pool Labelling

The microfluidic split and pool (S&P) system leverages microwell array technology that was developed for small molecule screening and microbial ecology (5, 6). However, for single-cell sequencing as taught in the instant application, no droplet merging or optical measurements are needed. This technology has been applied in conjunction with single-cell (SC) sequencing readouts in a different manner: for the stimulation of human cells before their recovery for SC sequencing (“StimDrop,”). StimDrop utilizes viable cells in emulsion droplets, whereas single-cell sequencing embodiments of the instant application utilize fixed and permeabilized cells. Handling of fixed and permeabilized cells for S&P barcoding as taught in the instant application uses an all-aqueous solution with low cellular dropout.

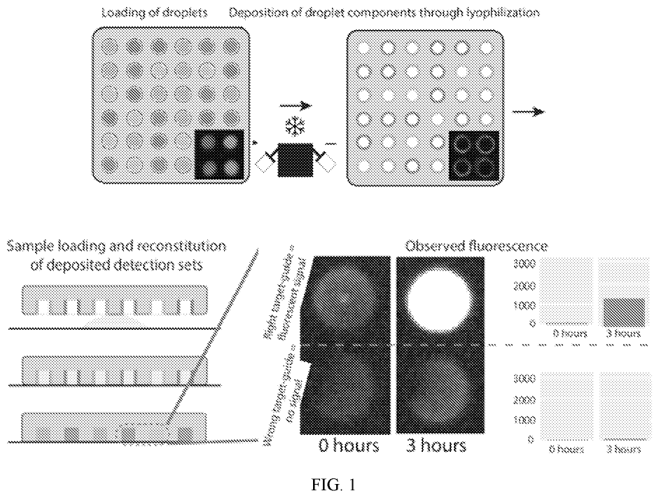

Barcodes are pre-loaded into multiple arrays using the published methods for loading droplet-borne reagents into microwells. This “factory” step is carried out cost-effectively ahead of time (optionally with automated liquid handling) for a large number of devices at once and the oligonucleotide barcodes dried down (and volatile oil removed) for stable long-term storage (FIG. 1). Then, fixed and permeabilized cells are introduced and S&P reactions take place by distributing the cells with buffer and enzyme across the microwell arrays, sealing the microwell arrays and reacting, then recovering cells from the microwell arrays and pooling them for the next of 2-3 “split” steps. In one embodiment, arrays with ˜100,000 microwells are used (accommodating 1,000,000+ cells) with similar barcode complexity (many microwells containing the same barcode) and cell density as published S&P work (27-29). Barcoded molecules of uniform length are then extracted from cells for more uniform PCR amplification and library construction using the robust short-read lab-on-chip systems to constitute an all-microfluidic processing system with minimal overall hands-on time for the assay and limited (optional) capital equipment. In another embodiment, increasing the number of barcodes enables even larger batch sizes without increasing the number of S&P steps and consequent cellular/molecular dropout.

### Example 2: High-Efficiency and Targeted RNA Tag Amplification Using Padlock Probes

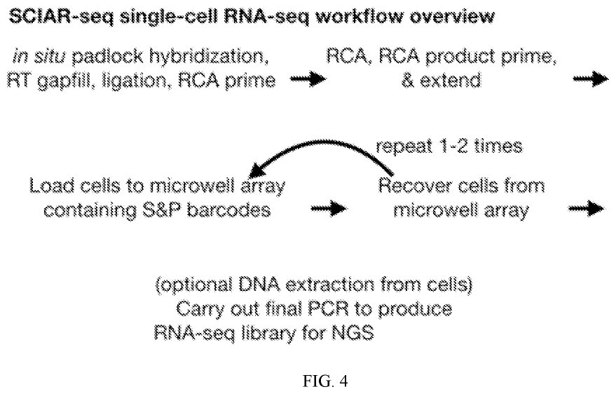

The instant disclosure provides a new molecular approach for SC RNA-seq sequence library construction, termed “SCIAR-seq” (Single-cell Combinatorial Indexing of Amplified RNAs), that works by pre-amplifying target RNA sequences of interest in situ followed by S&P barcoding. The targeted nature of SCIAR-seq in combination with linear signal amplification provides both a greatly enhanced sensitivity along with a massive reduction in the required sequencing for readout. In this scheme, padlock technology already in use is employed (FIG. 4).

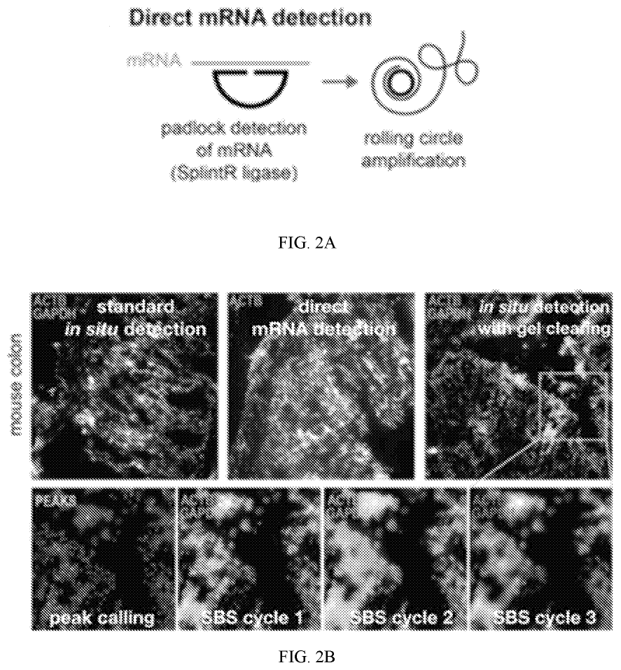

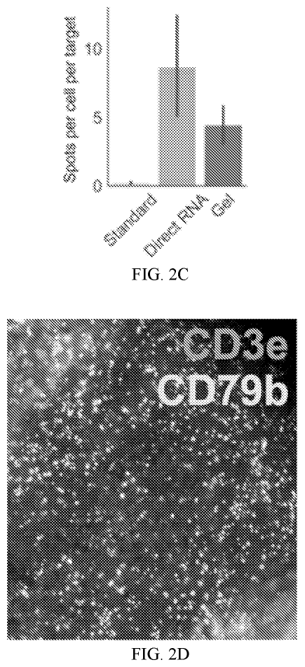

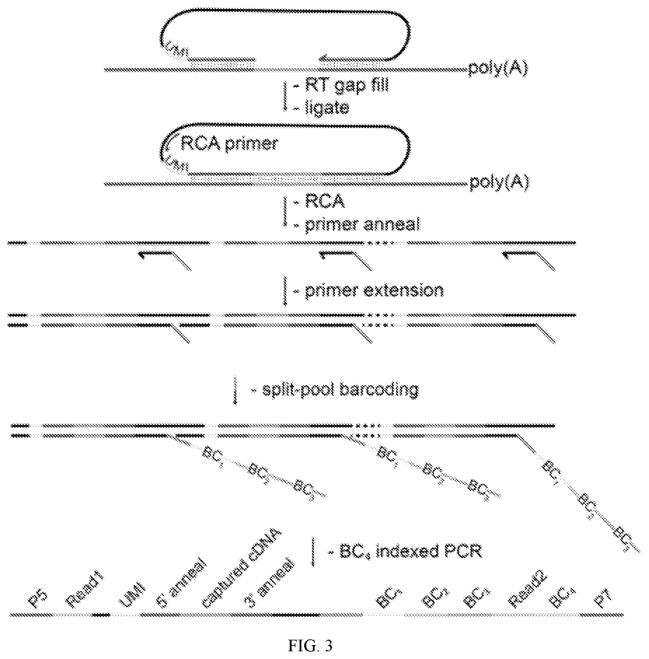

In one embodiment, SCIAR-seq is initialized by annealing padlock probes (32) directly to pre-defined RNA targets in situ with a gap between the arms of the probes (33) inside fixed and permeabilized cells (FIGS. 2A and 3). The production and use of large padlock libraries is well known (34-36). A non-strand-displacing reverse transcriptase fills in the gap and SplintR ligase (37) seals the resulting nick to form a DNA minicircle (FIG. 2B). This embodiment is an advance on the standard protocol for padlock fill-in and in situ sequencing on cDNA templates (FIGS. 2B through 2D)(38). The DNA mini-circles are of uniform length by design, and are primed and amplified linearly without excess dispersion (25, 26, 39) by rolling circle amplification (RCA) (40, 41). The RCA product is a linear single stranded DNA concatemer containing multiple copies of the synthetic padlock sequence, a UMI sequence, gene-specific hybridization sequences, and the nucleic acid sequence copied from the transcript (FIG. 2C). The RCA product is then primed on the ubiquitous padlock adaptor sequence and extended using a non-strand displacing DNA polymerase to create an array of double-stranded products containing a 5′ adaptor along with the padlock UMI and the targeted RNA sequence. Cells are subsequently subjected to S&P barcoding to append cell-specific barcode combinations to the targeted/amplified transcripts from each cell by overlap extension and/or ligation. The library is completed by appending platform-specific adaptors to the library molecules in a small number of PCR cycles (FIGS. 3 and 4).

SCIAR-seq enables transcripts of interest to be targeted for analysis. Target genes are each addressed by multiple padlocks to boost the accuracy and sensitivity of quantitation, potentially to 100%. Multiple padlocks per gene and pre-amplification before S&P reduce (gene-wise) molecular dropout through the S&P steps necessary to encode large batches of cells. Notably, SCIAR-seq is not susceptible to gene mis-identification resulting from erroneous probe hybridization events as this approach relies on sequence information copied directly from native RNA molecules. SCIAR-seq is also particularly well suited to handle large scale PERTURB-seq screens, as only one padlock is needed to capture the pool of gRNAs. In one embodiment, direct gRNA detection is employed. In another embodiment, the current generation of CROP-seq vectors provide synthetic gRNA-flanking adaptor sequences (42). Additionally, SCIAR-seq is targeted to read across splice junctions to get information about isoform distributions and assess allelic variation from analysis of the base sequences detected. Selecting targets of interest and tuning sensitivity based on the expected expression level, by variable multiplexing on large targets like genes, dramatically reduces the sequencing effort/cost required for readout. For example, in one embodiment wherein 250 selected targets are represented uniformly in a library, these targets are quantified with a precision of ˜30% (coefficient of variation modeling Poisson statistics) using only 2500 reads per cell, better serving many projects than the more than 50,000 non-targeted reads per cell needed in conjunction with extant methods of single-cell RNA sequencing.

If higher-quality information is obtained per cell, then fewer cells are needed to support a given analysis. In one embodiment, if a gene of interest is detected in only 5% of cells with standard approaches but can be detected in 50% of cells using SCIAR-seq, then only one-tenth the number of cells need to be processed, independent of the reductions cost per cell. The instant disclosure therefore provides an important synergistic benefit between per-cell data quality and overall cost.

In certain embodiments, even a modest reduction in sample preparation and sequencing requirement costs, for example 3× as taught by the instant disclosure, results in a total cost reduction of 10×3×3 or nearly 100×.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

### REFERENCES

1. Kim S, De Jonghe J, Kulesa A B, Feldman D, Vatanen T, Bhattacharyya R P, Berdy B, Gomez J, Nolan J, Epstein S, Blainey P C. High-throughput automated microfluidic sample preparation for accurate microbial genomics. Nat Commun. 2017; 8:13919. Epub 2017/01/28. doi: 10.1038/ncommsl3919. PubMed PMID: 28128213; PMCID: PMC5290157.
2. Hindson B, Saxonov S, Schnall-Levin M. Methods for droplet-based sample preparation. Google Patents; 2017.
3. Taber K A J, Dickinson B D, Wilson M. The promise and challenges of next-generation genome sequencing for clinical care. JAMA internal medicine. 2014; 174(2):275-80.
4. Reyes M, Vickers D, Billman K, Eisenhaure T, Hoover P, Browne E P, Rao D A, Hacohen N, Blainey P C. Multiplexed enrichment and genomic profiling of peripheral blood cells reveal subset-specific immune signatures. Science advances. 2019; 5(1):eaau9223.
5. Kulesa A, Kehe J, Hurtado J E, Tawde P, Blainey P C. Combinatorial drug discovery in nanoliter droplets. Proceedings of the National Academy of Sciences. 2018; 115(26):6685-90.
6. Kehe J, Kulesa A, Ortiz A, Ackerman C M, Thakku S G, Sellers D, Kuehn S, Gore J, Friedman J, Blainey P C. Massively parallel screening of synthetic microbial communities. Proceedings of the National Academy of Sciences. 2019; 116(26):12804-9.
7. Barczak A K, Gomez J E, Kaufmann B B, Hinson E R, Cosimi L, Borowsky M L, Onderdonk A B, Stanley S A, Kaur D, Bryant K F. RNA signatures allow rapid identification of pathogens and antibiotic susceptibilities. Proceedings of the National Academy of Sciences. 2012; 109(16):6217-22.
8. Lohr J G, Adalsteinsson V A, Cibulskis K, Choudhury A D, Rosenberg M, Cruz-Gordillo P, Francis J M, Zhang C-Z, Shalek A K, Satija R. Whole-exome sequencing of circulating tumor cells provides a window into metastatic prostate cancer. Nature biotechnology. 2014; 32(5):479.
9. Fan H C, Blumenfeld Y J, Chitkara U, Hudgins L, Quake S R. Noninvasive diagnosis of fetal aneuploidy by shotgun sequencing DNA from maternal blood. Proceedings of the National Academy of Sciences. 2008; 105(42):16266-71.
10. Beroud C, Karliova M, Bonnefont J, Benachi A, Munnich A, Dumez Y, Lacour B, Paterlini-Brechot P. Prenatal diagnosis of spinal muscular atrophy by genetic analysis of circulating fetal cells. The Lancet. 2003; 361(9362):1013-4.
11. Sparks A B, Struble C A, Wang E T, Song K, Oliphant A. Noninvasive prenatal detection and selective analysis of cell-free DNA obtained from maternal blood: evaluation for trisomy 21 and trisomy 18. American journal of obstetrics and gynecology. 2012; 206(4):319. e1-e9.
12. Kowarsky M, Camunas-Soler J, Kertesz M, De Vlaminck I, Koh W, Pan W, Martin L, Neff N F, Okamoto J, Wong R J. Numerous uncharacterized and highly divergent microbes which colonize humans are revealed by circulating cell-free DNA. Proceedings of the National Academy of Sciences. 2017; 114(36):9623-8.
13. Schwarzenbach H, Hoon D S, Pantel K. Cell-free nucleic acids as biomarkers in cancer patients. Nature Reviews Cancer. 2011; 11(6):426.
14. Adalsteinsson V A, Ha G, Freeman S S, Choudhury A D, Stover D G, Parsons H A, Gydush G, Reed S C, Rotem D, Rhoades J. Scalable whole-exome sequencing of cell-free DNA reveals high concordance with metastatic tumors. Nature communications. 2017; 8(1):1324.
15. De Vlaminck I, Valantine H A, Snyder T M, Strehl C, Cohen G, Luikart H, NeffNF, Okamoto J, Bernstein D, Weisshaar D. Circulating cell-free DNA enables noninvasive diagnosis of heart transplant rejection. Science translational medicine. 2014; 6(241):241ra77-ra77.
16. White R A, Blainey P C, Fan H C, Quake S R. Digital PCR provides sensitive and absolute calibration for high throughput sequencing. BMC genomics. 2009; 10(1):116.
17. Han J, Craighead H G. Separation of long DNA molecules in a microfabricated entropic trap array. Science (New York, N.Y.). 2000; 288(5468):1026-9.
18. Rozenblatt-Rosen O, Stubbington M J, Regev A, Teichmann S A. The human cell atlas: from vision to reality. Nature News. 2017; 550(7677):451.
19. Dixit A, Parnas O, Li B, Chen J, Fulco C P, Jerby-Arnon L, Marjanovic N D, Dionne D, Burks T, Raychowdhury R. Perturb-Seq: dissecting molecular circuits with scalable single-cell RNA profiling of pooled genetic screens. Cell. 2016; 167(7):1853-66. e17.
20. Rubin A J, Parker K R, Satpathy A T, Qi Y, Wu B, Ong A J, Mumbach M R, Ji A L, Kim D S, Cho S W. Coupled single-cell CRISPR screening and epigenomic profiling reveals causal gene regulatory networks. Cell. 2019; 176(1-2):361-76. e17.
21. Ranu N, Villani A-C, Hacohen N, Blainey P C. Targeting individual cells by barcode in pooled sequence libraries. Nucleic acids research. 2018; 47(1):e4-e.
22. Picelli S, Bjorklund A K, Faridani O R, Sagasser S, Winberg G, Sandberg R. Smart-seq2 for sensitive full-length transcriptome profiling in single cells. Nature methods. 2013; 10(11):1096.
23. Dal Molin A, Di Camillo B. How to design a single-cell RNA-sequencing experiment: pitfalls, challenges and perspectives. Briefings in bioinformatics. 2018.
24. Becskei A, Kaufmann B B, van Oudenaarden A. Contributions of low molecule number and chromosomal positioning to stochastic gene expression. Nature genetics. 2005; 37(9):937.
25. Chen C, Xing D, Tan L, Li H, Zhou G, Huang L, Xie X S. Single-cell whole-genome analyses by Linear Amplification via Transposon Insertion (LIANTI). Science (New York, N.Y.). 2017; 356(6334):189-94.
26. Shiroguchi K, Jia T Z, Sims P A, Xie X S. Digital RNA sequencing minimizes sequence-dependent bias and amplification noise with optimized single-molecule barcodes. Proceedings of the National Academy of Sciences. 2012; 109(4):1347-52.
27. Vitak S A, Torkenczy K A, Rosenkrantz J L, Fields A J, Christiansen L, Wong M H, Carbone L, Steemers F J, Adey A. Sequencing thousands of single-cell genomes with combinatorial indexing.

Nature methods. 2017; 14(3):302.
28. Cao J, Packer J S, Ramani V, Cusanovich D A, Huynh C, Daza R, Qiu X, Lee C, Furlan S N, Steemers F J. Comprehensive single-cell transcriptional profiling of a multicellular organism.

Science (New York, N.Y.). 2017; 357(6352):661-7.
29. Rosenberg A B, Roco C M, Muscat R A, Kuchina A, Sample P, Yao Z, Graybuck L T, Peeler D J, Mukherjee S, Chen W. Single-cell profiling of the developing mouse brain and spinal cord with split-pool barcoding. Science (New York, N.Y.). 2018; 360(6385):176-82.
30. Srivatsan S R, McFaline-Figueroa J L, Ramani V, Saunders L, Cao J, Packer J, Pliner H A, Jackson D L, Daza R M, Christiansen L. Massively multiplex chemical transcriptomics at single-cell resolution. Science (New York, N.Y.). 2020; 367(6473):45-51.
31. Datlinger P, Rendeiro A F, Boenke T, Krausgruber T, Barreca D, Bock C. Ultra-high throughput single-cell RNA sequencing by combinatorial fluidic indexing. bioRxiv. 2019.
32. Nilsson M, Malmgren H, Samiotaki M, Kwiatkowski M, Chowdhary B P, Landegren U.

Padlock probes: circularizing oligonucleotides for localized DNA detection. Science (New York, N.Y.). 1994; 265(5181):2085-8.
33. Ke R, Mignardi M, Pacureanu A, Svedlund J, Botling J, Wahlby C, Nilsson M. In situ sequencing for RNA analysis in preserved tissue and cells. Nature methods. 2013; 10(9):857-60. Epub 2013/07/16. doi: 10.1038/nmeth.2563. PubMed PMID: 23852452.
34. Hardenbol P, Baner J, Jain M, Nilsson M, Namsaraev E A, Karlin-Neumann G A, Fakhrai-Rad H, Ronaghi M, Willis T D, Landegren U. Multiplexed genotyping with sequence-tagged molecular inversion probes. Nature biotechnology. 2003; 21(6):673.
35. Turner E H, Lee C, Ng S B, Nickerson D A, Shendure J. Massively parallel exon capture and library-free resequencing across 16 genomes. Nature methods. 2009; 6(5):315.
36. Zhang K, Gore A. Designing padlock probes for targeted genomic sequencing. Google Patents; 2014.
37. Lohman G J, Zhang Y, Zhelkovsky A M, Cantor E J, Evans Jr T C. Efficient DNA ligation in DNA-RNA hybrid helices by Chlorella virus DNA ligase. Nucleic acids research. 2013; 42(3):1831-44.
38. Feldman D, Singh A, Schmid-Burgk J L, Carlson R J, Mezger A, Garrity A J, Zhang F, Blainey P C. Optical pooled screens in human cells. Cell. 2019; 179(3):787-99. e17.
39. Zong C, Lu S, Chapman A R, Xie X S. Genome-wide detection of single-nucleotide and copy-number variations of a single human cell. Science (New York, N.Y.). 2012; 338(6114):1622-6.
40. Lizardi P M, Huang X, Zhu Z, Bray-Ward P, Thomas D C, Ward D C. Mutation detection and single-molecule counting using isothermal rolling-circle amplification. Nature genetics. 1998; 19(3):225.
41. Larsson C, Koch J, Nygren A, Janssen G, Raap A K, Landegren U, Nilsson M. In situ genotyping individual DNA molecules by target-primed rolling-circle amplification of padlock probes. Nature methods. 2004; 1(3):227.
42. Datlinger P, Rendeiro A F, Schmidl C, Krausgruber T, Traxler P, Klughammer J, Schuster L C, Kuchler A, Alpar D, Bock C. Pooled CRISPR screening with single-cell transcriptome readout. Nature methods. 2017; 14(3):297.

1. A method for performing single-cell nucleic acid sequencing upon cells of a tissue sample, the method comprising:
(i) obtaining a tissue sample from a subject;
(ii) permeabilizing cells of the tissue sample;
(iii) contacting the permeabilized cells of the tissue sample with a padlock probe comprising a sequence complementary to a target nucleic acid sequence, thereby producing a padlock probe bound to the target nucleic acid sequence;
(iv) contacting the treated cells with a reverse transcriptase and/or a polymerase, thereby capturing the target nucleic acid sequence on the padlock probe;
(v) contacting the target nucleic acid sequence on the padlock probe with ligase, thereby circularizing the padlock probe having the target nucleic acid sequence;
(vi) performing rolling circle amplification (RCA) upon the circularized padlock probe, thereby creating a linear repeating sequence (LRS) comprising the target nucleic acid sequence;
(vii) contacting said LRS with a primer comprising an LRS complement sequence and an index adaptor sequence;
(viii) subjecting the treated cells of the tissue sample to combinatorial indexing, thereby generating an extended primer comprising the LRS complement sequence, the adaptor sequence, and a barcode sequence capable of identifying the cell of origin; and
(ix) identifying a polynucleotide sequence of the extended primer;
thereby obtaining single cell nucleic acid sequencing data from the tissue sample.

2. The method of claim 1, wherein the target nucleic acid sequence comprises a target RNA sequence or complement thereof.

3. The method of claim 1, wherein the padlock probe comprises a unique molecular identifier (UMI), optionally wherein the UMI is between 8 and 20 nucleotides in length.

4. The method of claim 1, wherein the barcode sequence is between 6 and 20 nucleotides in length.

5. The method of claim 1, wherein the target nucleic acid sequence comprises a RNA sequence, optionally a RNA sequence selected from the group consisting of a mRNA, a snRNA, a lcRNA, a siRNA and a gRNA.

6. The method of claim 1, wherein the target nucleic acid sequence comprises a mRNA or other nucleic acid sequence selected from a pathway and/or gene of FIG. 6.

7. The method of claim 1, wherein the target nucleic acid sequence comprises a DNA barcode sequence, optionally wherein the DNA barcode sequence identifies and/or is attached to an antibody, optionally wherein detection of the DNA barcode sequence identifies antibody abundance and/or levels of a protein bound by the antibody, optionally wherein the method is performed to quantify target protein levels in a CITE-Seq and/or REAP-Seq process.

8. The method of claim 1, wherein the combinatorial indexing is applied in combination with cell splitting, optionally wherein the combinatorial indexing is applied in combination with between 1 and 10 iterations of cell splitting.

9. The method of claim 1, wherein the combinatorial indexing comprises use of a microfluidic chamber.

10. The method of claim 1, wherein the RCA is performed by a DNA polymerase.

11. The method of claim 1, wherein single cell nucleic acid sequencing data is obtained from between about 1,000,000 and about cells 1×10¹² in a single run.

12. The method of claim 1, wherein the target nucleic acid sequence is present at less than ten copies in a single cell, optionally less than nine copies in a single cell, optionally less than eight copies in a single cell, optionally less than seven copies in a single cell, optionally less than six copies in a single cell, optionally less than five copies in a single cell, optionally less than four copies in a single cell, optionally less than three copies in a single cell, optionally less than two copies in a single cell, optionally at one copy in a single cell.

13. The method of claim 1, wherein the polymerase is a non-strand displacing DNA polymerase, optionally selected from the group consisting of Q5® High-Fidelity DNA Polymerase, Phusion® High-Fidelity DNA Polymerase, KAPA HiFi DNA Polymerase, Pfu DNA polymerase, KOD DNA polymerase, T4 DNA polymerase, T7 DNA polymerase and an exonuclease deficient variant of Taq (TaqIT).

14. An improved method for obtaining quantitative nucleic acid sequence data, the method comprising:
(i) contacting a sample comprising a target nucleic acid sequence with a padlock probe comprising a sequence complementary to the target nucleic acid sequence, thereby generating a padlock probe bound to the target nucleic acid sequence;
(ii) contacting the padlock probe bound to the target nucleic acid sequence with a reverse transcriptase and/or a polymerase, thereby capturing the target nucleic acid sequence on the padlock probe;
(iii) contacting the target nucleic acid sequence on the padlock probe with ligase, thereby circularizing the padlock probe having the target nucleic acid sequence;
(iv) performing rolling circle amplification (RCA) upon the circularized padlock probe, thereby creating a linear repeating sequence (LRS) comprising the target nucleic acid sequence; and
(v) identifying a sequence of the LRS and optionally correlating the sequence of the LRS with a single target nucleic acid and/or single cell of origin,
thereby obtaining quantitative nucleic acid sequence data.

15. The method of claim 14, wherein the padlock probe comprises a unique molecular identifier (UMI), optionally wherein the UMI is between 8 and 20 nucleotides in length.

16. The method of claim 14, wherein the target nucleic acid sequence is a target RNA sequence, optionally wherein the target nucleic acid is selected from the group consisting of a mRNA, a snRNA, a lcRNA, a siRNA and a gRNA.

17. The method of claim 14, wherein the target nucleic acid sequence comprises a mRNA or other nucleic acid sequence selected from a pathway and/or gene of FIG. 6.

18. The method of claim 14, wherein the target nucleic acid sequence comprises a DNA barcode sequence, optionally wherein the DNA barcode sequence identifies and/or is attached to an antibody, optionally wherein detection of the DNA barcode sequence identifies antibody abundance and/or levels of a protein bound by the antibody, optionally wherein the method is performed to quantify target protein levels in a CITE-Seq and/or REAP-Seq process.

19. The method of claim 14, wherein:
the RCA is performed by a DNA polymerase;
single cell nucleic acid sequencing data is obtained from between about 1,000,000 and about cells 1×10¹² in a single run;
the polymerase is a non-strand displacing DNA polymerase, optionally selected from the group consisting of Q5@ High-Fidelity DNA Polymerase, Phusion® High-Fidelity DNA Polymerase, KAPA HiFi DNA Polymerase, Pfu DNA polymerase, KOD DNA polymerase, T4 DNA polymerase, T7 DNA polymerase and an exonuclease deficient variant of Taq (TaqIT);
and/or the target nucleic acid sequence is of low abundance in the sample comprising the target nucleic acid sequence.

20-22. (canceled)

23. A composition selected from the group consisting of:
A composition comprising a plurality of padlock probes targeting two or more genes and/or RNAs selected from FIG. 6; and
A kit comprising a plurality of padlock probes targeting two or more genes and/or RNAs selected from FIG. 6 and instructions for its use.

24. (canceled)
